(19) United States
(12) Patent Application Publication
(10) Pub. No.: US 2026/0183571 A1
(43) Pub. Date: Jul. 2, 2026

(54) BEAM DISTRIBUTION DETERMINATION METHOD, STORAGE MEDIUM, AND BEAM DISTRIBUTION DETERMINATION SYSTEM

(71) Applicant: Shanghai United Imaging Healthcare Co., Ltd., Shanghai (CN)

(72) Inventor: Xiaoliang XIA, Shanghai (CN)

(73) Assignee: Shanghai United Imaging Healthcare Co., Ltd., Shanghai (CN)

(21) Appl. No.: 19/433,292

(22) Filed: Dec. 26, 2025

(30) Foreign Application Priority Data
Dec. 26, 2024 (CN) 202411954403.X
Dec. 19, 2025 (CN) 202511935298.X

(51) Int. Cl. A61N 5/10

(52) U.S. Cl. CPC A61N 5/1065; A61N 5/1071

(57) ABSTRACT

A beam distribution determination method includes: determining at least one beam distribution component based on a measured dose distribution of a beam to be modeled, where dimensionality of parameter space required to parametrically model the at least one beam distribution component is lower than dimensionality of parameter space required to parametrically model a beam distribution of the beam to be modeled; and determining a target beam distribution of the beam to be modeled based on the at least one beam distribution component.

### CROSS-REFERENCE TO RELATED APPLICATIONS

This application claims priority to Chinese Patent Application No. 202411954403.X, filed on Dec. 26, 2024, and Chinese Patent Application No. 202511935298.X filed on Dec. 19, 2025. The disclosures of the aforementioned applications are hereby incorporated by reference in their entireties.

### TECHNICAL FIELD

The present disclosure relates to the field of medical technologies, and in particular, to a beam distribution determination method, a storage medium, and a beam distribution determination system.

### BACKGROUND

In numerous technical fields such as physics and engineering, a spatial distribution problem of a beam or a field from a source is frequently encountered. Herein, the beam or the field may be widely understood as a spatial distribution of a physical quantity such as energy, particles, light waves, or sound waves. For example, in radiation therapy, a beam generated by a treatment head is usually designed as a regular circular cross-section. However, due to a number of unavoidable reasons, an actual beam deviates, more or less, from the circular shape.

Currently, in order to model a beam deviating from the circular shape, usually each process in the treatment head is simulated. However, computational efficiency of this modeling approach is extremely low.

### SUMMARY

In view of above, embodiments of the present disclosure aim to provide a beam distribution determination method, a storage medium, and a beam distribution determination system, so as to solve a problem of low efficiency in calculating a beam distribution in the related art.

In a first aspect, the present disclosure provides a beam distribution determination method, including: determining at least one beam distribution component based on a measured dose distribution of a beam to be modeled, where dimensionality of parameter space required to parametrically model the at least one beam distribution component is lower than dimensionality of parameter space required to parametrically model a beam distribution of the beam to be modeled; and determining a target beam distribution of the beam to be modeled based on the at least one beam distribution component.

In a second aspect, the present disclosure further provides a beam distribution determination method, including: acquiring a measured dose distribution of a beam to be modeled; and determining a target beam distribution of the beam to be modeled based on the measured dose distribution, the target beam distribution including at least one one-dimensional beam distribution component.

In a third aspect, the present disclosure further provides a non-transitory computer-readable storage medium storing a computer program, and when the computer program is executed by a processor, steps of the beam distribution determination method described above are implemented.

In a fourth aspect, the present disclosure further provides a beam distribution determination system, including: a processor; a memory, storing executable instructions of the processor. The processor is configured to implement the following steps: determining at least one beam distribution component based on a measured dose distribution of a beam to be modeled, where dimensionality of parameter space required to parametrically model the at least one beam distribution component is lower than dimensionality of parameter space required to parametrically model a beam distribution of the beam to be modeled; and determining a target beam distribution of the beam to be modeled based on the at least one beam distribution component.

In the beam distribution determination method mentioned in the embodiments of the present disclosure, by determining a finite quantity of beam distribution components using the measured dose distribution of the beam to be modeled, an objective of converting complex multi-dimensional modeling into modeling of the finite quantity of beam distribution components with a lower dimensionality is achieved. As a result, in the embodiments of the present disclosure, the dimensionality of parameter space of modeling is reduced, so that workload of modeling and parameter tuning is significantly reduced, and calculation efficiency is improved.

### BRIEF DESCRIPTION OF THE DRAWINGS

FIG. 1 is a schematic diagram of an application scenario according to an embodiment of the present disclosure.

FIG. 2 is a schematic flowchart of a beam distribution determination method according to an embodiment of the present disclosure.

FIG. 3 is a schematic diagram of a location relationship between a specific two-dimensional plane and a beam source according to an embodiment of the present disclosure.

FIG. 4A is a schematic flowchart of determining at least one basis function based on a measured dose distribution of a beam to be modeled according to an embodiment of the present disclosure.

FIG. 4B is a schematic flowchart of determining at least one basis function based on a measured dose distribution of a beam to be modeled according to another embodiment of the present disclosure.

FIG. 5 is a schematic flowchart of determining a target beam distribution of a beam to be modeled based on at least one basis function according to an embodiment of the present disclosure.

FIG. 6 is a schematic flowchart of parameterizing at least one basis function according to an embodiment of the present disclosure.

FIG. 7A and FIG. 7B are schematic diagrams of beam modeling results according to an embodiment of the present disclosure.

FIG. 8A and FIG. 8B are schematic diagrams of beam modeling results according to another embodiment of the present disclosure.

FIG. 9A and FIG. 9B are schematic diagrams of beam modeling results according to still another embodiment of the present disclosure.

FIG. 10 is a schematic flowchart of determining a target beam distribution according to an embodiment of the present disclosure.

FIG. 11 is a schematic flowchart of a beam distribution determination method according to another embodiment of the present disclosure.

FIG. 12 is a hardware structure block diagram of a terminal of a beam distribution determination method according to an embodiment of the present disclosure.

FIG. 13 is a schematic flowchart of a beam distribution determination method according to an embodiment of the present disclosure.

FIG. 14 is a schematic flowchart of a beam distribution determination method according to another embodiment of the present disclosure.

FIG. 15 is a schematic diagram of a one-dimensional distribution of a circular beam according to an optional embodiment of the present disclosure.

FIG. 16 is a schematic diagram of a two-dimensional distribution of a circular beam according to an optional embodiment of the present disclosure.

FIG. 17 is a schematic diagram of a one-dimensional distribution of two reference directions of a non-circular beam according to an optional embodiment of the present disclosure.

FIG. 18 is a schematic diagram of a synthesized two-dimensional distribution of a non-circular beam according to an optional embodiment of the present disclosure.

FIG. 19 is a schematic diagram of a one-dimensional distribution of two reference directions of a non-circular beam according to another optional embodiment of the present disclosure.

FIG. 20 is a schematic diagram of a synthesized two-dimensional distribution of a non-circular beam according to another optional embodiment of the present disclosure.

FIG. 21 is a schematic flowchart of a beam distribution determination method according to still another embodiment of the present disclosure.

FIG. 22 is a schematic structural diagram of an intelligent agent according to an embodiment of the present disclosure.

FIG. 23 is a schematic structural diagram of a beam distribution determination system according to an embodiment of the present disclosure.

FIG. 24 is a schematic structural diagram of a radiation therapy device according to an embodiment of the present disclosure.

### DETAILED DESCRIPTION OF THE EMBODIMENTS

The following clearly and completely describes technical solutions in embodiments of the present disclosure with reference to accompanying drawings in the embodiments of the present disclosure. Apparently, the described embodiments are merely some but not all of embodiments of the present disclosure. All other embodiments obtained by those skilled in the art based on the embodiments of the present disclosure without creative efforts shall fall within the protection scope of the present disclosure.

Before the embodiments of the present disclosure are described, technical terms that may be mentioned in the embodiments of the present disclosure are explained and described firstly.

Radiation therapy: refers to a clinical technique that uses various types of radiation (such as X-rays, electron beams, and proton beams) to destroy tumor tissue. The embodiments of the present disclosure are mainly used for a pre-treatment plan design and a verification stage of radiation therapy.

Beam: refers to a beam of radiation emitted from a radiation therapy device (for example, a linear accelerator treatment head) for treatment in the field of radiation therapy. The embodiments of the present disclosure provide an efficient and accurate mathematical description (that is, modeling) of characteristics of the beam in space.

Beam distribution: specifically refers to a function defined on a two-dimensional plane and representing spatial variation of relative intensity or particle flux of the beam within the two-dimensional plane in the embodiments of the present disclosure. It is one of objectives of the embodiments of the present disclosure to efficiently and accurately model the function.

Treatment Planning System (TPS): refers to a clinical system that integrates software and hardware. One of its core functions is to perform dose calculation using an accurate beam distribution model. A beam distribution determination method provided in the embodiments of the present disclosure may be used as a key functional module of the system, to improve modeling efficiency and precision of an irregular beam.

A core principle of radiation therapy is to use high-energy beams to accurately irradiate a target area in an organism. Energy is deposited in biological tissues through the beams to form a specific dose distribution, so as to destroy tumor cells while maximally protecting surrounding normal tissues. In this process, an accurate beam distribution model is a key basis for the treatment planning system to perform dose calculation and ensure treatment effect and safety.

In a treatment planning system, a dose calculation algorithm requires the beam distribution as a core input parameter. According to differences between specific algorithm models, the beam distribution may be represented as a distribution of particle flux, energy flux, kerma flux, and the like in space.

For a beam with circular symmetry in an ideal state, the beam distribution can be simplified to a one-dimensional function ƒ(r) related only to an off-axis distance r. A modeling process in the following case is efficient. First, a value of ƒ(r) is measured and determined based on a series of discrete r values to form a data table, and then a continuous one-dimensional function is reconstructed by using an interpolation algorithm. In this way, a beam distribution value of any point (x, y) on a two-dimensional plane may be obtained by means of calculation of r=\sqrt{x²+y²} and substitution into ƒ(r). Under this model, the core of modeling is to adjust and optimize discrete values of the one-dimensional function ƒ(r) repeatedly until a dose calculation result matches measured data. Because this involves only parameter tuning of the one-dimensional function, workload thereof is usually within an acceptable range.

However, an actual beam distribution commonly deviates from the ideal circular symmetry due to inter-axial asymmetry of an accelerator tube, energy dispersion caused by deflection of an electron beam in a magnetic field, non-uniformity of a target material, and impact of a non-circular component such as a primary collimator. Irregular shapes such as ellipse shape, rounded triangle, and pebble shape are usually presented. In such an asymmetric case, the beam distribution can no longer be simplified to a one-dimensional function ƒ(r), but needs to rely on a two-dimensional function ƒ(x, y) for a complete description.

If a modeling pathway of representing the function as a discrete data table is adopted, ƒ(x, y) needs to be sampled and adjusted on a two-dimensional grid. Assuming that N points are sampled in each of directions x and y, a quantity of parameters needing to be adjusted surges to an order of N². This modeling method requires an exponentially increased workload, compared with the one-dimensional case requiring only N parameters, and consumes huge computing resources, and a debugging process is extremely complex. As a result, modeling efficiency is low, costs are high, and it is difficult to meet urgent requirements for fast and accurate modeling in clinical applications. This severely restricts optimization efficiency of treatment planning and clinical application efficacy of the system.

To solve the technical problems described above, the embodiments of the present disclosure provide a beam distribution determination method, a storage medium, and a beam distribution determination system. The beam distribution determination method includes: determining at least one beam distribution component based on a measured dose distribution of a beam to be modeled, where dimensionality of parameter space required to parametrically model the at least one beam distribution component is lower than dimensionality of parameter space required to parametrically model a beam distribution of the beam to be modeled; and determining a target beam distribution of the beam to be modeled based on the at least one beam distribution component.

In the beam distribution determination method mentioned in the embodiments of the present disclosure, by determining a finite quantity of beam distribution components using the measured dose distribution of the beam to be modeled, an objective of converting complex multi-dimensional modeling into modeling of the finite quantity of beam distribution components with a lower dimensionality is achieved. As a result, in the embodiments of the present disclosure, the dimensionality of parameter space of modeling is reduced, so that workload of modeling and parameter tuning is significantly reduced, and calculation efficiency is improved.

It should be noted that the reduced dimensionality or the lower dimensionality mentioned in the embodiments of the present disclosure primarily refers to a reduction in the dimensionality of parameter space. The dimensionality of parameter space refers to a total quantity of continuous or discrete parameters that require independent adjustment during the modeling process. For conventional methods that directly adjust intensity values on a two-dimensional planar grid, the dimensionality of parameter space equals a quantity of grid points (e.g., an order of N×N). The embodiments of the present disclosure, however, decompose the beam distribution into a finite quantity of beam distribution components (e.g., one-dimensional reference distributions in specific directions, or separable one-dimensional radial functions from basis functions). Consequently, only these corresponding one-dimensional functions require parameterization. This approach drastically reduces the number of parameters requiring adjustment to a scale related to a quantity of one-dimensional sampling points (e.g., k×M, where k is a quantity of components and M is a quantity of sampling points on each one-dimensional function). The dimensionality of parameter space is substantially lower, thereby achieving an order-of-magnitude improvement in modeling efficiency.

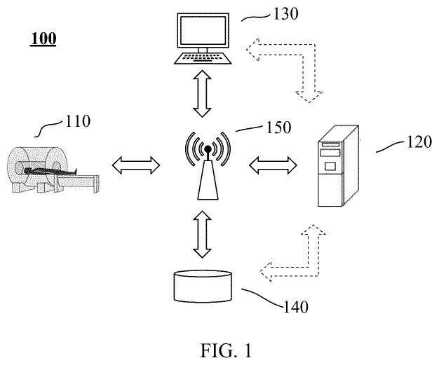

An example application scenario of the present disclosure is described below with reference to FIG. 1.

FIG. 1 is a schematic diagram of an application scenario according to an embodiment of the present disclosure. As shown in FIG. 1, the application scenario includes a radiation therapy device 110, a processing device 120, an interaction device 130, and a storage device 140. These components may perform data exchange through a network 150, and cooperate together to complete beam modeling and therapy tasks.

The radiation therapy device 110 is a core apparatus for generating therapeutic radiation (that is, a beam). It has two main modes of operation. In a beam modeling phase, the radiation therapy device 110 outputs, based on a control instruction, a beam to be modeled for data collection. In a treatment execution phase, the radiation therapy device 110 outputs a precise beam to an organism (for example, a patient) based on a treatment plan.

As a computing core, the processing device 120 is configured to execute the beam distribution determination method mentioned in the embodiments of the present disclosure. For example, the processing device 120 may implement the following steps: determining at least one beam distribution component based on a measured dose distribution of a beam to be modeled, where dimensionality of parameter space required to parametrically model the at least one beam distribution component is lower than dimensionality of parameter space required to parametrically model a beam distribution of the beam to be modeled; and determining a target beam distribution of the beam to be modeled based on the at least one beam distribution component. The target beam distribution is used as a key input parameter and is provided to a subsequent dose calculation algorithm (for example, a treatment planning system), to generate or verify a treatment plan. The processing device 120 may be at least one of a Central Processing Unit (CPU), a Digital Signal Processor (DSP), a System-On-Chip (SOC), or a Micro-Control Unit (MCU). In some embodiments, the processing device 120 or a portion of the processing device 120 may be integrated into the radiation therapy device 110.

The interaction device 130 provides an interaction interface for a user, and is a device for the user to interact with the radiation therapy device 110 and the processing device 120. Optionally, the interaction device 130 may include a keyboard, a mouse, a touchscreen, a voice input device, and the like. For example, the user may enter an instruction via a keyboard, perform operation selection via a mouse, perform a touch gesture operation on a touchscreen, or speak an instruction via a voice input device, to interact with the radiation therapy device 110 and the processing device 120. For example, the user inputs the measured dose distribution of the beam to be modeled through the interactive device 130. The interaction device 130 includes a display screen that may display received data. For example, the interaction device may present a target beam distribution generated by the processing device 120 for the user to view, confirm, or adjust accordingly. Optionally, the interaction device 130 may further include a virtual interaction device, for example, a Virtual Reality (VR) device or an Augmented Reality (AR) device. In some embodiments, the interaction device 130 may be integrated into the processing device 120.

The storage device 140 may store data and/or instructions. Optionally, the storage device 140 may store the target beam distribution obtained from the processing device 120, input data obtained from the interaction device 130, and the like. In some embodiments, the storage device 140 may store data and/or instructions that may be executed by the processing device 120 or are used to perform the exemplary method in the present disclosure. Optionally, the storage device 140 includes a mass storage device, a removable storage device, a volatile read/write memory, a read-only memory, and the like. In some embodiments, the storage device 140 may be integrated into the processing device 120.

The network 150 provides a medium for communication between the foregoing components, and may be a wired network or a wireless network. It should be noted that a connection relationship between components in the figure is merely an example. In some embodiments, some components (for example, the processing device 120, the interaction device 130, and the storage device 140) can communicate with each other in a direct connection or system bus mode to improve data exchange efficiency.

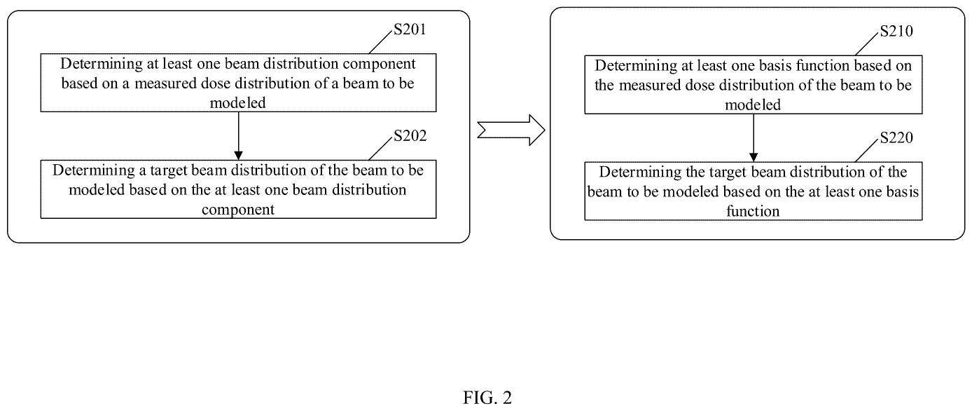

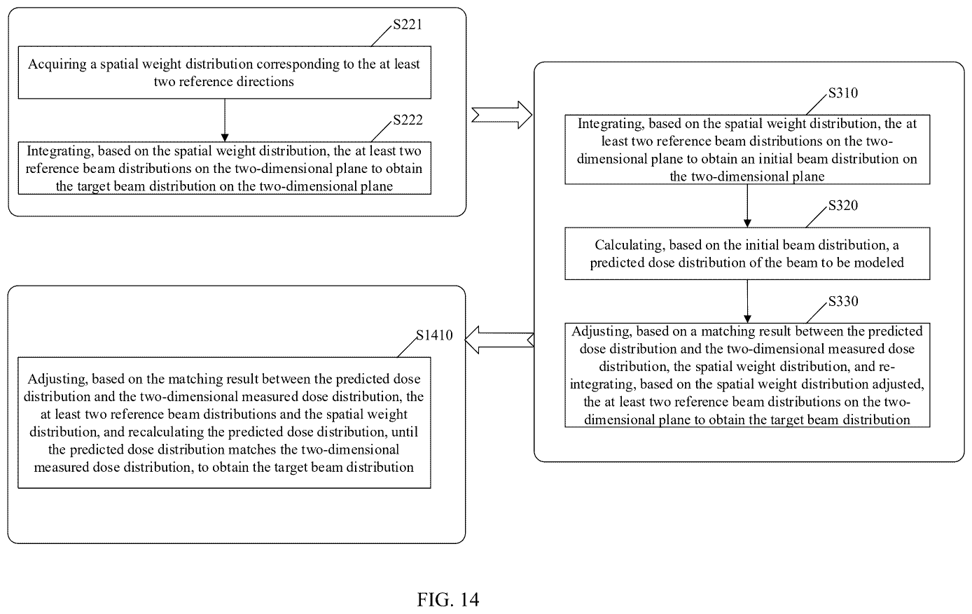

After the example application scenario in the embodiment of the present disclosure is briefly described, the following describes in detail beam distribution determination methods provided in the embodiments of the present disclosure with reference to FIG. 2 to FIG. 21.

FIG. 2 is a schematic flowchart of a beam distribution determination method according to an embodiment of the present disclosure. For example, the beam distribution determination method provided in this embodiment of the present disclosure is executed by a processor in the radiation therapy device 110 shown in FIG. 1. As shown in FIG. 2, the beam distribution determination method provided in this embodiment of the present disclosure includes the following steps.

Step S201: determining at least one beam distribution component based on a measured dose distribution of a beam to be modeled.

The measured dose distribution refers to spatial distribution data obtained by actually detecting, by using a radiation measurement device (such as an ion chamber array or an Electronic Portal Imaging Device (EPID)), a radiation dose deposited by the beam to be modeled in a specific spatial region. The specific spatial region may be one or more preset two-dimensional planes (namely, a specific two-dimensional plane), and correspondingly a two-dimensional measured dose distribution is obtained. The specific spatial region may alternatively be a three-dimensional space, and correspondingly a three-dimensional measured dose distribution is obtained.

Dimensionality of the at least one beam distribution component is lower than dimensionality of a beam distribution of the beam to be modeled. That is, dimensionality of parameter space of the at least one beam distribution component required to parametrically model is lower than dimensionality of parameter space required to parametrically model the beam distribution of the beam to be modeled. The beam distribution of the beam to be modeled is a complete beam distribution characterized by the measured dose distribution. In other words, a quantity of independent parameters required for parametric modeling of the at least one beam distribution component is significantly less than a quantity of parameters required for parametric modeling of the complete beam distribution characterized by the measured dose distribution. Alternatively, in the embodiments of the present disclosure, the dimensionality of parameter space required for iterative search in a modeling optimization process is reduced by using the at least one beam distribution component as an intermediate representation. This means that optimization algorithms need to search for solutions in a smaller and simpler parameter space, significantly reducing computational burden and improving modeling efficiency.

For example, the beam distribution component may include, but is not limited to, the following forms. In a first form, at least two reference directions are determined based on shape characteristics of the measured dose distribution, to obtain a reference beam distribution corresponding to each reference direction, which is taken as the beam distribution component. The reference beam distribution is a one-dimensional function. In a second form, a beam distribution function describing the measured dose distribution is decomposed into a plurality of basis functions in a polar coordinate system or a spherical coordinate system; and then at least one objective basis function may be selected from the plurality of basis functions as the beam distribution component. The basis function includes a combination of a one-dimensional radial function and an angle function.

Step S202: determining a target beam distribution of the beam to be modeled based on the at least one beam distribution component.

The target beam distribution is a continuous function expression of the measured dose distribution and is a key input for subsequent dose calculation. In other words, processing such as combination (for example, linear summation), parameterization, and iterative optimization is performed on the basis function, to finally obtain a mathematical model that can predict the measured dose distribution with high accuracy, that is, the target beam distribution.

In the beam distribution determination method mentioned in the embodiments of the present disclosure, by determining a finite quantity of beam distribution components using the measured dose distribution of the beam to be modeled, an objective of converting complex multi-dimensional modeling into modeling of the finite quantity of beam distribution components with a lower dimensionality is achieved. As a result, in the embodiments of the present disclosure, the dimensionality of parameter space of modeling is reduced, so that workload of modeling and parameter tuning is significantly reduced, and calculation efficiency is improved.

Still referring to FIG. 2, the following example illustrates a case where the beam distribution component is a basis function.

In the embodiments of the present disclosure, the determining at least one beam distribution component based on the measured dose distribution of the beam to be modeled (step S201), includes step S210.

Step S210: determining at least one basis function based on the measured dose distribution of the beam to be modeled.

The at least one basis function is used to represent a multi-dimensional function corresponding to the beam distribution of the beam to be modeled. That is, the at least one basis function can be converted into the multi-dimensional function corresponding to the beam distribution of the beam to be modeled. The multi-dimensional function includes, but is not limited to, a two-dimensional function, a three-dimensional function, or even a more-dimensional function. Representing a complex multi-dimensional beam distribution by using a combination of a limited quantity of basis functions is a key to implementing dimensionality reduction modeling in this embodiment of the present disclosure. Each basis function may have a relatively simple mathematical form. In an example, the basis function may include a combination of a one-dimensional radial function and an angle function, which will be described in detail in subsequent embodiments.

It can be understood that under certain specific conditions, the basis function may include a one-dimensional function. Specifically, as a fundamental mathematical unit for constructing the target beam distribution through linear combination, the basis function is determined through function decomposition (such as Fourier series decomposition or spherical harmonic decomposition) in a coordinate system used (such as polar coordinate system or spherical coordinate system), and is mainly presented in the following two forms. A first form is a combination of a one-dimensional radial function and an angle function. A second form is a basis function corresponding to zero order in function decomposition with an angle function part as a constant (such as circularly or spherically symmetric fundamental components). The second form is a basis function degenerating into a one-dimensional radial function. The second form may be regarded as a special case of the first form. Therefore, in the embodiment of the present disclosure, the basis function may include one or more one-dimensional radial functions (such as a zero-order basis function), as well as product of one or more one-dimensional radial functions and angle functions (such as non-zero order basis functions).

In the embodiment of the present disclosure, the determining the target beam distribution of the beam to be modeled based on the at least one beam distribution component (step S202), includes step S220.

Step S220: determining the target beam distribution of the beam to be modeled based on the at least one basis function.

Therefore, in the beam distribution determination method mentioned in this embodiment of the present disclosure, by determining a finite quantity of basis functions using the measured dose distribution of the beam to be modeled, an objective of converting complex multi-dimensional modeling into modeling of the finite quantity of basis functions is achieved. As a result, in the embodiments of the present disclosure, the dimensionality of parameter space of modeling is reduced, so that workload of modeling and parameter tuning is significantly reduced, and calculation efficiency is improved.

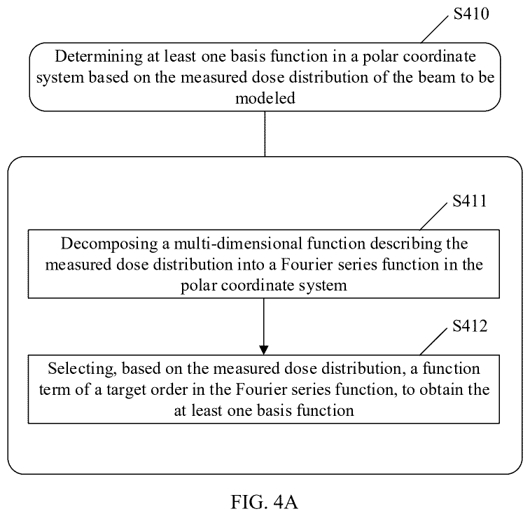

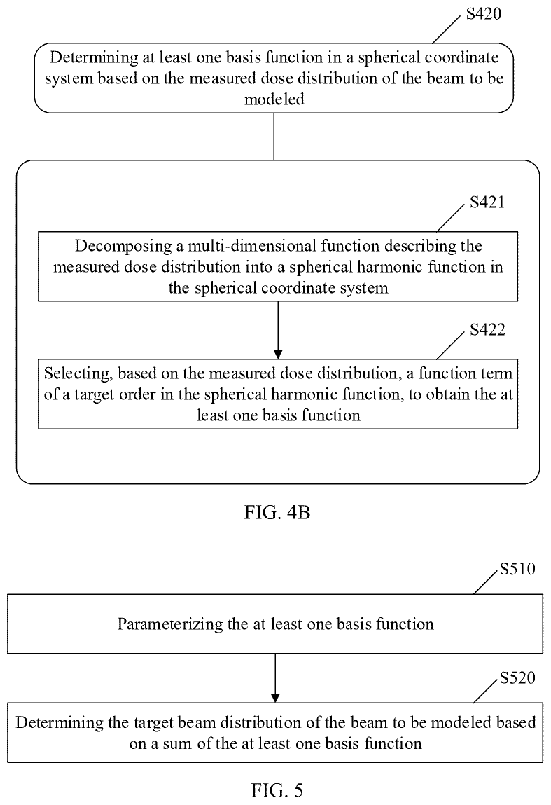

With reference to FIG. 4A and FIG. 4B, the following separately describes how to specifically determine the at least one basis function based on the measured dose distribution of the beam to be modeled.

FIG. 4A is a schematic flowchart of determining at least one basis function based on a measured dose distribution of a beam to be modeled according to an embodiment of the present disclosure. The embodiment shown in FIG. 4A is extended based on the embodiment shown in FIG. 2. The following focuses on a difference between the embodiment shown in FIG. 4A and the embodiment shown in FIG. 2, and similarities are not described again.

As shown in FIG. 4A, in this embodiment of the present disclosure, the determining the at least one basis function based on the measured dose distribution of the beam to be modeled (step S210) includes the following steps.

Step S410: determining at least one basis function in a polar coordinate system based on the measured dose distribution of the beam to be modeled.

In this embodiment of the present disclosure, the measured dose distribution includes two-dimensional measured dose distribution on a two-dimensional plane. The two-dimensional plane may include one or more preset two-dimensional planes (namely, a specific two-dimensional plane). A location of the specific two-dimensional plane (for example, a distance and a direction relative to a beam source) may be preset. For example, the specific two-dimensional plane may be perpendicular to a beam center line, or may have a specific included angle with a plane perpendicular to the beam center line. This is not specifically limited in this embodiment of the present disclosure. Accordingly, the measured dose distribution is a measured dose distribution on the specific two-dimensional plane. The shape characteristics of the measured dose distribution can be reflected by a shape of its isodose lines or morphology of dose points.

In one implementation, to maximize modeling efficiency and reduce data processing costs, a measured dose distribution on only one specific two-dimensional plane (for example, an isocentric plane) may be measured. This approach has a small calculation amount, can quickly complete modeling, and is especially applicable to a routine clinical treatment scenario that has a high requirement on a modeling speed.

In another implementation, in order to improve modeling accuracy to adapt to beams with complex spatial characteristics, a measured dose distribution of the beam to be modeled can be measured on a plurality of different specific two-dimensional planes (for example, a plurality of specific two-dimensional planes distributed along a beam propagation path), thereby capturing a distribution variation law of the beam to be modeled along a propagation direction.

After the measured dose distribution is obtained, a shape (namely, a shape characteristic) of the measured dose distribution is analyzed, and based on the shape of the measured dose distribution, the measured dose distribution is represented in a functional form including at least one basis function in a polar coordinate system. The basis function is a basic mathematical unit for describing and reconstructing the measured dose distribution, and is essentially to decompose and represent a beam distribution characteristic of the beam to be modeled. Each of a part or all of the basis functions includes a one-dimensional radial function and an angle function. The one-dimensional radial function is a one-dimensional function that varies only with a polar radius and is independent of a polar angle. The one-dimensional radial function is used to represent a distribution of beam intensity or flux along a radial direction. The angle function is a function that varies only with the polar angle and is independent of the polar radius. The angle function is used to represent a variation characteristic of the beam distribution in different angular directions, reflecting asymmetry of the beam.

After the basis function is determined, a beam distribution function in the polar coordinate system may be constructed by using a linear combination of the basis functions, and is denoted as ƒ(r,θ). Therein, r represents a distance from a coordinate point to a pole, and θ represents an included angle between a line connecting the coordinate point and the pole and a polar axis. The pole may be any point on a specific two-dimensional plane on which the measured dose distribution is located, for example, an intersection point between a beam center line and the specific two-dimensional plane may be used as the pole.

Subsequently, parameter tuning and optimization are performed for the beam distribution function ƒ(r,θ) to make a distribution morphology described by the beam distribution function consistent with an actual measured dose distribution. A specific optimization process may include: (1) performing discrete sampling for a one-dimensional radial function in each basis function within its domain; (2) initializing or adjusting function values of these one-dimensional radial functions at each discrete sampling point; (3) using an interpolation algorithm (such as linear interpolation or spline interpolation) to calculate a continuous function value of the one-dimensional radial function at any polar radius r based on the function values of the discrete sampling points; (4) combining an interpolated one-dimensional radial function and a corresponding angle function, to obtain, by calculation, a value of the beam distribution function ƒ(r,θ) at any polar coordinate point (r,θ); (5) inputting the beam distribution function ƒ(r,θ) into a dose calculation algorithm (for example, a treatment planning system) to obtain a predicted dose distribution; (6) comparing the predicted dose distribution with the actual measured dose distribution; (7) based on a comparison result, iteratively adjusting a function value of the one-dimensional radial function at a discrete point, and repeating steps (3) to (6); (8) ending the iteration when an error between the predicted dose distribution and the measured dose distribution is less than a preset threshold. In this case, the beam distribution expressed by a combination of the basis functions finally determined is determined as the target beam distribution.

According to the beam distribution determination method mentioned in this embodiment of the present disclosure, the measured dose distribution of the beam to be modeled is used to determine a finite quantity of basis functions in the polar coordinate system, where each basis function includes a one-dimensional radial function and an angle function, thereby achieving an objective of converting complex two-dimensional modeling into modeling a finite quantity of one-dimensional functions. As a result, in the embodiments of the present disclosure, the dimensionality of parameter space of modeling is reduced, so that workload of modeling and parameter tuning is significantly reduced, and calculation efficiency is improved. In addition, in this embodiment of the present disclosure, by introducing the angle function (for example, a cosine function) into the basis function, mathematical description capability for an irregular beam shape such as an ellipse shape or a rounded triangle may be retained, thereby ensuring accuracy and flexibility of modeling.

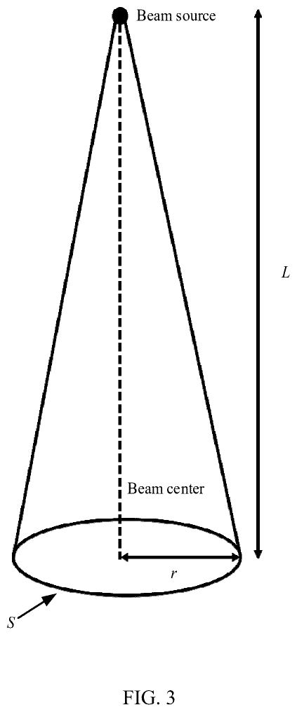

With reference to FIG. 3, the following describes a specific implementation of determining a two-dimensional measured dose distribution on a two-dimensional plane.

FIG. 3 is a schematic diagram of a location relationship between a two-dimensional plane and a beam source according to an embodiment of the present disclosure. As shown in FIG. 3, a beam is emitted from a beam source and spreads in an approximately conical shape in space. A center axis of the beam (that is, a beam center line) is represented by the dashed line in FIG. 3, S is a two-dimensional plane perpendicular to the beam center line, and L represents a distance (that is, an axial distance) from the beam source to the two-dimensional plane S.

Based on a principle of radiophysics, when the distance L is large enough, a beam distribution in space (which can be represented as particle flux, energy flux, kerma, and the like) is inversely proportional to the square of L. Therefore, beam distributions on other two-dimensional planes in space can be derived after a beam distribution on only one specific two-dimensional plane is determined.

Specifically, it is assumed that the beam distribution on the specific two-dimensional plane is represented as ƒ(x, y, L₀), where (x, y) represents rectangular coordinates on the two-dimensional plane, and L₀ represents a distance from the beam source to the specific two-dimensional plane. In this case, for any two-dimensional plane that is perpendicular to the beam center line and whose distance is L in front of the beam source, a beam distribution ƒ(x, y, L) on the two-dimensional plane may be determined by using the following formula (1).

$$f ( x , y , L ) = f ( \frac{{L}_{0} x}{L} , \frac{{L}_{0} y}{L} , {L}_{0} ) \times \frac{{L}_{0}^{2}}{{L}^{2}} \qquad ( 1 )$$

Based on this, in this embodiment of the present disclosure, modeling of a three-dimensional beam distribution is first mapped to a two-dimensional plane for processing, thereby significantly simplifying calculation complexity.

In a practical application process, first, in a selected specific two-dimensional plane, dose values of a plurality of discrete measurement points are obtained by using a radiation measurement device. These measurement points together form a two-dimensional measured dose distribution on the specific two-dimensional plane. The specific two-dimensional plane is preferably perpendicular to the beam center line, and the distance L₀ between the specific two-dimensional plane and the beam source may be flexibly set based on a model of a radiation therapy device, a calibration specification, and a requirement of a clinical treatment scenario. For example, the distance may usually be set to 100 cm, 150 cm, or the like.

After a high-precision two-dimensional measured dose distribution is obtained, a target beam distribution of the beam to be modeled can be determined based on the two-dimensional measured dose distribution.

In this embodiment of the present disclosure, dose data is collected on the specific two-dimensional plane, thereby achieving an objective of effectively condensing beam distribution characteristics in three-dimensional space into a two-dimensional measured dose distribution. The two-dimensional measured dose distribution fully presents a dose distribution pattern of a radiation field on the specific two-dimensional plane, and lays a reliable data basis for efficient and accurate inversion calculation of the target beam distribution based on embodiments of the present disclosure. This further provides an accurate beam model input for dose calculation of a treatment plan.

Still referring to FIG. 4A, in some embodiments, the determining the at least one basis function in the polar coordinate system based on the measured dose distribution of the beam to be modeled (step S410) may include the following steps.

Step S411: decomposing a multi-dimensional function describing the measured dose distribution into a Fourier series function in the polar coordinate system.

In some embodiments, the multi-dimensional function describing the measured dose distribution refers to a function that depends on a plurality of spatial variables and is used to represent the measured dose distribution. The multi-dimensional function may be defined as a function form ƒ(x, y) in a rectangular coordinate system, or may be converted into an equivalent form ƒ(r,θ) in the polar coordinate system. ƒ(x, y) and ƒ(r, θ) satisfy a conversion relationship expressed by the following formula (2).

$$\{ \begin{matrix}x = r cos θ \\ y = r sin θ\end{matrix} \qquad ( 2 )$$

Then, the multi-dimensional function ƒ(r,θ) is expressed in a Fourier series form in the polar coordinate system, to obtain the following formula (3).

$$f ( r , θ ) = {\sum }_{n = 0}^{∞} {f}_{n} ( r ) cos [ n ( θ - {ψ}_{n} ) ] \qquad ( 3 )$$

In formula (3), n represents an order, and n is an integer; ƒₙ(r) is a one-dimensional radial function of the n^{th} order; and Un represents a phase angle of a cosine function of the n^{th} order.

Based on identity transformation of a trigonometric function, the multi-dimensional function ƒ(r, θ) may also be expressed as the following formula (4) or formula (5).

$$f ( r , θ ) = {\sum }_{n = 0}^{∞} {f}_{n} ( r ) sin [ n ( θ - {ϕ}_{n} ) ] \qquad ( 4 )$$

$$f ( r , θ ) = {\sum }_{n = 0}^{∞} {g}_{n} ( r ) cos ( n θ ) + {h}_{n} ( r ) sin ( n θ ) \qquad ( 5 )$$

In formula (4), φₙ represents a phase angle of a sine function of the n^{th} order. In formula (5), each of the gₙ(r) and hₙ(r) is a one-dimensional radial function of the n^{th} order.

It should be noted that the Fourier series may alternatively be in another mathematical equivalent form, which may be determined by those skilled in the art based on a function relationship. This is not specifically limited in this embodiment of the present disclosure.

Step S412: selecting, based on the measured dose distribution, a function term of a target order in the Fourier series function, to obtain the at least one basis function.

A selection basis for the target order includes: the function term corresponding to the target order makes a significant contribution to describing a shape of the measured dose distribution. Specifically, intensity of a harmonic component in an angular direction of the measured dose distribution (that is, coefficient or weight corresponding to the function term of each order) may be analyzed to determine these orders with significant contributions. In practical application, key target orders may also be directly selected based on prior knowledge of a beam shape (for example, it is known that the beam is ellipse-shaped or rounded triangular).

The Fourier series function in the polar coordinate system is used as an example. An expansion form of the Fourier series function may be the following formula (6).

$$f ( r , θ ) = {f}_{0} ( r ) + {f}_{1} ( r ) cos ( θ - {ψ}_{1} ) + {f}_{2} ( r ) cos [ 2 ( θ - {ψ}_{2} ) ] + {f}_{3} ( r ) cos [ 3 ( θ - {ψ}_{3} ) ] + … \qquad ( 6 )$$

In formula (6), ƒ₀(r) is a function term of the zeroth-order, ƒ₁(r) cos(θ−ψ₁) is a function term of the first-order, ƒ₂(r) cos[2(θ−ψ₂)] is a function term of the second-order, and so forth.

For example, to simplify calculation, the target orders may include the zeroth order and the first order. Function terms of the target orders are ƒ₀(r) and ƒ₁(r) cos(θ−φ₁), respectively. In this case, ƒ₀(r) and ƒ₁(r) cos(θ−φ₁) may be respectively used as basis functions. The zeroth-order function term ƒ₀(r) is a one-dimensional radial function, and the first-order function term ƒ₁(r) cos(θ−φ₁) is a combination of a one-dimensional radial function ƒ₁(r) and an angle function cos(θ−φ₁).

Alternatively, to improve modeling accuracy, the target orders may include the zeroth order, the first order, and the second order. Function terms of the target orders are ƒ₀(r), ƒ₁(r) cos(θ−ψ₁), and ƒ₂(r) cos[2(θ−ψ₂)] respectively. In this case, ƒ₀(r), ƒ₁(r) cos(θ−ψ₁), and ƒ₂(r) cos[2(θ−ψ₂)] may be respectively used as the basis functions. The second-order function term ƒ₂(r) cos[2(θ−ψ₂)] is a combination of a one-dimensional radial function ƒ₂(r) and an angle function cos[2(θ−ψ₂)].

In the beam distribution determination method mentioned in this embodiment of the present disclosure, the multi-dimensional function corresponding to the measured dose distribution is first decomposed into a Fourier series form in the polar coordinate system, that is, mathematically expressed as a combination of a one-dimensional radial function and an angle function of the infinite order. Then, a finite quantity of target order terms are selected as basis functions. In this way, a theoretically infinite-dimensional complex problem is reduced to a simplified model including only the finite quantity of basis functions. Through dimensionality reduction processing, key characteristics of beams are accurately retained, and modeling complexity and calculation workload are greatly reduced, significantly improving beam modeling efficiency.

In the foregoing embodiment, the target order is determined by comprehensively considering modeling efficiency and modeling accuracy. The following describes a specific implementation of determining the target order with examples.

In some embodiments, the target order is determined based on the shape of the measured dose distribution of the beam to be modeled. The following is considered: the shape of the measured dose distribution often exhibits specific symmetries or periodicities, such as a special form like an ellipse shape, a rounded triangular, or an pebble shape. It should be noted that the description of the shape herein is intended to visually convey its visual characteristics, and does not refer to a geometric shape that is mathematically strictly defined.

In an actual application process, an operator or a computer may observe a shape characteristic of the measured dose distribution, and use, based on a correlation between the shape characteristic and each function term in the Fourier series function, at least one order with highest correlation as the target order. A quantity of target orders may be determined based on shape complexity of the measured dose distribution and required modeling accuracy. This is not specifically limited in this embodiment of the present disclosure.

A correspondence between the shape characteristic of the measured dose distribution and the target order is described below with examples.

For an ellipse-shaped measured dose distribution, it exhibits a biaxially symmetric characteristic. Based on this, it is determined that the target orders include the zeroth order and the second order, that is, n=0 and n=2. The zeroth-order function term provides a circular symmetric basis, and the second-order function term is used to describe eccentricity and a direction of an ellipse.

Still referring to the Fourier series function in the foregoing embodiment, basis functions corresponding to the ellipse-shaped measured dose distribution are ƒ₀(r) and ƒ₂(r) cos[2(θ-ψ₂)], respectively. Assuming that a long axis of the ellipse shape is an x-axis on a two-dimensional plane, a phase angle is 0, that is, ψ₂=0.

For a rounded triangular measured dose distribution, it has three symmetry axes with an angle of 120° between adjacent symmetry axes. Based on this, it is determined that the target orders include the zeroth order and the third order, that is, n=0 and n=3. The zeroth-order function term provides a circular symmetric basis, and the third-order function term is used to generate convex morphology of three symmetrical distributions.

Still referring to the Fourier series function in the foregoing embodiment, basis functions corresponding to the rounded triangular measured dose distribution are ƒ₀(r) and ƒ₃(r) cos[3(θ-ψ₃)], respectively. Assuming that the three symmetry axes are respectively located in directions of 90°, 210°, and 330° on a plane, a phase angle is 90°, that is, ψ₃=90°.

For a pebble-shaped measured dose distribution, one end is blunt, and the other end is sharp. Based on this, it is determined that the target orders include the zeroth order and the first order and the third order, that is, n=0, n=1, and n=3. The zeroth-order function term provides a circular symmetric basis, the first-order function term is used to create an offset between the blunt end and the sharp end and break axial symmetry, and the third-order function term is used to further modify the blunt end and the sharp end.

Still referring to the Fourier series function in the foregoing embodiment, basis functions corresponding to the pebble-shaped measured dose distribution are ƒ₀(r), ƒ₁(r) cos(θ-ψ₁), and ƒ₃(r) cos[3(θ-ψ₃)], respectively. Assuming that the sharp end of the pebble shape faces a positive direction of a y-axis on the two-dimensional plane, a phase angle is 90°, that is ψ₁=ψ₃=90°.

It should be noted that the foregoing correspondence between the shape characteristic of the measured dose distribution and the target order is merely an example. Those skilled in the art may select different target orders for measured dose distributions of different shapes according to their experiences, and apply the beam distribution determination method mentioned in this embodiment of the present disclosure to a modeling process of a beam distribution of another shape.

The embodiments of the present disclosure achieve a direct mapping from physical intuition-based shapes to mathematical models. A finite quantity of orders that contributes most significantly to the shape are selected to ensure accuracy and efficiency of model construction. Therefore, while a capability of describing a core shape is retained, a quantity of model parameters and complexity of parameter tuning are greatly reduced, thereby achieving an excellent balance between modeling accuracy and efficiency.

FIG. 4B is a schematic flowchart of determining at least one basis function based on a measured dose distribution of a beam to be modeled according to another embodiment of the present disclosure. The embodiment shown in FIG. 4B is extended based on the embodiment shown in FIG. 2. The following focuses on a difference between the embodiment shown in FIG. 4B and the embodiment shown in FIG. 2, and similarities are not described again.

As shown in FIG. 4B, in this embodiment of the present disclosure, the determining the at least one basis function based on the measured dose distribution of the beam to be modeled (step S210) includes the following steps.

Step S420: determining at least one basis function in a spherical coordinate system based on the measured dose distribution of the beam to be modeled.

In this embodiment of the present disclosure, the measured dose distribution includes a three-dimensional measured dose distribution in a three-dimensional space. That is, the measured dose distribution is spatial distribution data that is directly collected by a radiation measurement device in a three-dimensional spatial space and reflects a dose deposition characteristic of the beam to be modeled. Unlike measurement made only on a single two-dimensional plane, three-dimensional measurement aims to obtain complete dose variation information of the beam to be modeled along its direction of propagation and cross-sections at different locations in the direction. Similarly, in this embodiment of the present disclosure, each of a part or all of the at least one basis function includes a one-dimensional radial function and an angle function.

The three-dimensional measured dose distribution can represent spatial morphology of the beam to be modeled more comprehensively, and especially can capture complex characteristics of attenuation of a dose distribution in a longitudinal direction, a scattering effect, and a change in beam profile shape with a depth. This information is critical for a dose calculation algorithm that requires extremely high accuracy. As a result, the embodiments of the present disclosure achieve an objective of constructing a three-dimensional beam distribution model that is mathematically concise and faithful to physical authenticity based on real and complex three-dimensional physical measurement data.

Still referring to FIG. 4B, in some embodiments, the determining the at least one basis function in the spherical coordinate system based on the measured dose distribution of the beam to be modeled (step S420) may include the following steps.

Step S421: decomposing a multi-dimensional function describing the measured dose distribution into a spherical harmonic function in the spherical coordinate system.

For example, a multi-dimensional function ƒ(r, θ, φ) describing a three-dimensional measured dose distribution is decomposed into a series of spherical harmonic functions (that is, a spherical harmonic expansion) in the spherical coordinate system. Correspondingly, in the spherical coordinate system, the basis function may be represented by the following formula (7).

$${B}_{n , l , m} ( r , θ , ϕ ) = {R}_{n} ( r ) {Y}_{l}^{m} ( θ , ϕ ) \qquad ( 7 )$$

In formula (7), n represents a radial order, l represents an angular quantum number or an orbital angular momentum quantum number, m represents a magnetic quantum number, r represents a radial distance (also referred to as a radius), θ represents a polar angle, and φ represents an azimuth. Rₙ(r) is a one-dimensional radial function, which changes only with the radial distance r, and is used to represent a distribution of beam intensity or flux along the radial direction.

$${Y}_{l}^{m} ( θ , ϕ )$$

is a spherical harmonic function (that is, an angle function), which changes only with the polar angle θ and the azimuth angle φ, and is used to represent a change characteristic of the beam distribution in different angle directions in a three-dimensional space, so as to completely describe three-dimensional spatial asymmetry of the beam.

Therefore, in this embodiment of the present disclosure, the basis function is a basic mathematical unit for describing and reconstructing the three-dimensional measured dose distribution, and its essence is to decompose and represent a distribution characteristic of the beam to be modeled in the three-dimensional space. After the three-dimensional measured dose distribution is obtained, a spatial configuration (that is, a three-dimensional shape characteristic) is analyzed, and based on the shape characteristic, the function describing the dose distribution is represented as a combination of a finite quantity of the foregoing basis functions in the spherical coordinate system.

Step S422: selecting, based on the measured dose distribution, a function term of a target order in the spherical harmonic function, to obtain the at least one basis function.

In this embodiment of the present disclosure, a selection basis for the target order is as follows: the function term corresponding to the target order contributes significantly to description of a spatial shape (the three-dimensional configuration) of the three-dimensional measured dose distribution. Intensity of harmonic components in the three-dimensional dose distribution in various directions on the sphere may be analyzed to determine these orders and a power number with significant contributions. In practical application, the target order may be directly selected based on prior knowledge of beam three-dimensional shapes.

Considering that a three-dimensional shape of an actual beam distribution has specific physical properties and symmetry (for example, spherical symmetry, axial symmetry, or a specific petal structure), its spatial configuration is not completely random. Therefore, infinite-series spherical harmonic function terms are not needed, and only function terms corresponding to a finite critical target order need to be selected as basis functions to achieve high-accuracy representation.

A correspondence between the spatial shape characteristic of the three-dimensional measured dose distribution and the target order of the spherical harmonic function will be described in the following with an example.

For a nearly spherically symmetric three-dimensional measured dose distribution, its spatial configuration is highly symmetric, and a dose value mainly changes with the radius r. In this case, a spherical harmonic function term of order 1 is mainly selected as the basis function. For a three-dimensional measured dose distribution that is axially symmetric and ellipsoidal (for example, elongated or flattened along a beam flow axis), it has rotational symmetry about an axis, but varies in a direction of the polar angle θ. In this case, spherical harmonic function terms of orders 1 and 2 are mainly selected for combination.

Therefore, a beam distribution function in the spherical coordinate system may be constructed by selecting basis functions corresponding to a finite quantity of orders for combination. Subsequently, by parameterizing and iteratively optimizing each basis function, distribution morphology described by the combination is consistent with an actual three-dimensional measured dose distribution, and finally the target beam distribution is obtained.

The beam distribution determination method mentioned in this embodiment of the present disclosure uses the three-dimensional measured dose distribution of the beam to be modeled to determine a finite quantity of basis functions, including one-dimensional radial functions and spherical harmonic functions (angle functions) in the spherical coordinate system, thereby achieving an objective of converting complex three-dimensional modeling into modeling a finite quantity of one-dimensional radial functions. This also greatly reduces the dimensionality of parameter space of modeling, so that workload of modeling and parameter tuning is significantly reduced, and calculation efficiency is improved.

It may be understood that the foregoing embodiments are mainly described based on the polar coordinate system and the spherical coordinate system, but the technical solutions of the present disclosure are not limited thereto. Any coordinate system in which a multi-dimensional function describing a beam distribution can be decomposed into a form of a product of a one-dimensional radial function and an angle function in a variable separation manner is applicable to the beam distribution determination method mentioned in the embodiments of the present disclosure. For example, for a beam with obvious ellipsoidal symmetry, an elliptic coordinate system may be used and a Mathieu function may be used for decomposition. For a beam that changes smoothly along an axial direction, a cylindrical coordinate system may be used for processing. Those skilled in the art may select an appropriate coordinate system for decomposition based on a specific shape characteristic of a beam distribution. A core idea is to reduce dimensionality of a high-dimensional modeling problem to a finite quantity of one-dimensional radial functions for processing.

In the foregoing embodiments, specific implementations of determining a basis function based on a measured dose distribution are described. With reference to FIG. 5, the following further describes how to determine a target beam distribution based on a basis function.

FIG. 5 is a schematic flowchart of determining a target beam distribution of a beam to be modeled based on at least one basis function according to an embodiment of the present disclosure. The embodiment shown in FIG. 5 is extended based on the embodiment shown in FIG. 2. The following focuses on a difference between the embodiment shown in FIG. 5 and the embodiment shown in FIG. 2, and similarities are not described again.

As shown in FIG. 5, in this embodiment of the present disclosure, the determining the target beam distribution of the beam to be modeled based on the at least one basis function (step S220) may include the following steps.

Step S510: parameterizing the at least one basis function.

For example, discrete sampling is performed on a one-dimensional radial function in any basis function, and a function value of the one-dimensional radial function is adjusted at each sampling point location. Then, function values of the one-dimensional radial function are adjusted based on a plurality of sampling point locations by interpolation or the like, to obtain a function value of the one-dimensional radial function at any location point. Furthermore, the function values of the one-dimensional radial function are substituted into the basis function to achieve parameterization of the basis function.

Step S520: determining the target beam distribution of the beam to be modeled based on a sum of the at least one basis function.

In an implementation, a sum of the at least one basis function may be directly determined as the target beam distribution.

For example, for an ellipse-shaped measured dose distribution, the target beam distribution may be expressed by the following formula (8).

$$f ( r , θ ) = {f}_{0} ( r ) + {f}_{2} ( r ) cos ( 2 θ ) \qquad ( 8 )$$

For example, for a rounded triangular measured dose distribution, the target beam distribution may be expressed by the following formula (9).

$$f ( r , θ ) = {f}_{0} ( r ) + {f}_{3} ( r ) cos [ 3 ( θ - 90 ° ) ] \qquad ( 9 )$$

For example, for a pebble-shaped measured dose distribution, the target beam distribution may be expressed by the following formula (10).

$$f ( r , θ ) = {f}_{0} ( r ) + {f}_{1} ( r ) cos ( θ - 90 ° ) + {f}_{3} ( r ) cos [ 3 ( θ - 90 ° ) ] \qquad ( 10 )$$

Alternatively, the target beam distribution may be obtained by generalizing a linear combination of basis functions. For example, a constant term or a coordinate-dependent offset function may be added to a summation expression of basis functions to compensate for a description deviation of the basis functions from the measured dose distribution. Alternatively, weight coefficients may be assigned to the basis functions in a sum expression of the basis functions, so as to adjust a degree of contribution of the basis functions to overall morphology.

In this embodiment of the present disclosure, a complex two-dimensional distribution modeling problem is decomposed into parameter tuning of a finite quantity of one-dimensional radial functions, and then a complete distribution is restored by using a combination of basis functions, so as to implement conversion from a parameter tuning of a two-dimensional discrete grid to parameter tuning of a finite quantity of one-dimensional functions. Therefore, this greatly reduces the dimensionality of parameter space of modeling, so that workload of sampling and iterative optimization is significantly reduced, and overall efficiency of beam modeling is improved.

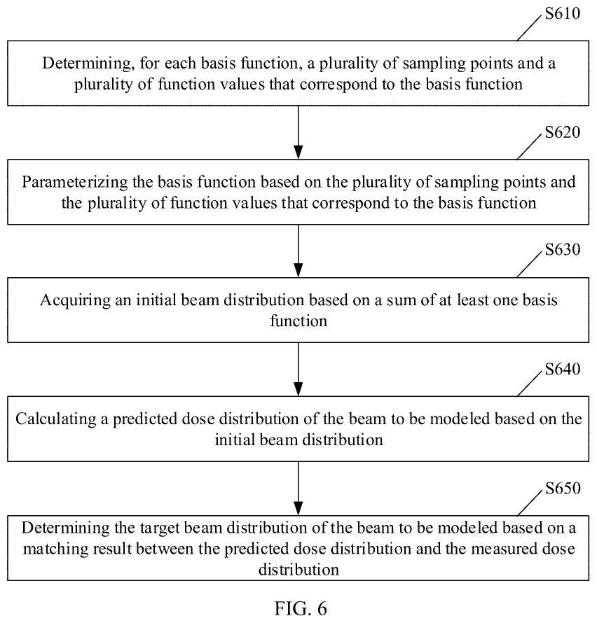

With reference to FIG. 6, the following further describes how to determine a target beam distribution based on a basis function.

FIG. 6 is a schematic flowchart of parameterizing at least one basis function according to an embodiment of the present disclosure. The embodiment shown in FIG. 6 is extended based on the embodiment shown in FIG. 5. The following focuses on a difference between the embodiment shown in FIG. 6 and the embodiment shown in FIG. 5, and similarities are not described again.

As shown in FIG. 6, in this embodiment of the present disclosure, the parameterizing the at least one basis function (step S510) may include the following steps.

Step S610: determining, for each basis function, a plurality of sampling points and a plurality of function values that correspond to the basis function.

The plurality of sampling points are in a one-to-one correspondence with the plurality of function values.

Specifically, for a one-dimensional radial function ƒₙ(r) in each basis function, a series of discrete sampling points r₁, r₂, r₃, . . . , rₖ are selected from a domain of the one-dimensional radial function, and a function value corresponding to each sampling point is determined separately, to obtain ƒₙ(r₁), ƒₙ(r₂), ƒₙ(r₃), . . . , ƒₙ(rₖ). A quantity of sampling points, a distribution spacing between sampling points, and the like may be determined according to an actual requirement. This is not specifically limited in this embodiment of the present disclosure.

Step S620: parameterizing the basis function based on the plurality of sampling points and the plurality of function values that correspond to the basis function.

Step S620 is intended to represent a continuous one-dimensional radial function by discrete sampled data. Specifically, a discrete data set of the sampling points and the function values in the foregoing steps is processed in an interpolation manner or a data fitting manner to obtain a correspondence between a sampling point and a function value. In this way, a function value of the one-dimensional radial function at any location may be determined. Substituting the function value into the basis function achieves parameterization of the basis function.

Step S630: acquiring an initial beam distribution based on a sum of the at least one basis function.

For example, the sum of the at least one basis function may be directly used as the initial beam distribution, or an offset function may be appropriately added on a basis of the summation, or a weight coefficient may be assigned to the basis function, to enhance model flexibility. A difference between the initial beam distribution and the target beam distribution lies in that the initial beam distribution may be an assumed model in an iteration during an iterative optimization process, whereas the target beam distribution is a model that is finally determined after iterative optimization is complete and that can accurately reflect a physical characteristic of an actual beam.

Step S640: calculating a predicted dose distribution of the beam to be modeled based on the initial beam distribution.

For example, the initial beam distribution is used as an input for dose calculation software, and a predicted dose distribution corresponding to the initial beam distribution is obtained through calculation by using a dose calculation method.

Step S650: determining the target beam distribution of the beam to be modeled based on a matching result between the predicted dose distribution and the measured dose distribution.

For example, the matching result can represent different information between the predicted dose distribution and the measured dose distribution, and the difference information may be quantized by using a value of an objective function (also referred to as a loss function). The objective function includes, but is not limited to, a root mean square error, a gamma pass rate, and the like.

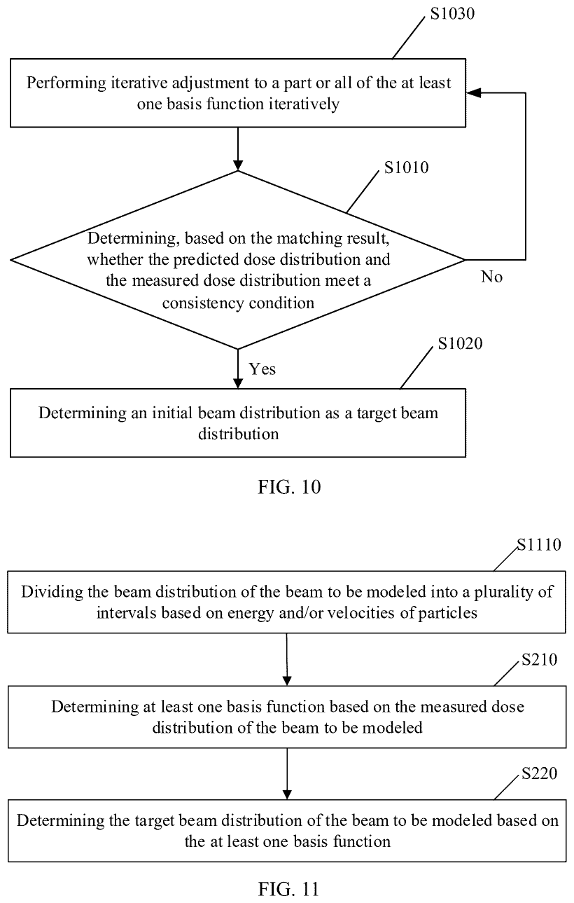

In some embodiments, the predicted dose distribution is quantitatively compared with the measured dose distribution to obtain a matching result, and then judgement is performed based on the matching result, so as to finally determine the target beam distribution. Specifically, if the matching result indicates that a difference between the predicted dose distribution and the measured dose distribution is large, the initial beam distribution is updated by returning to step S610 to adjust function values of one or more one-dimensional radial functions at sampling points, and steps S640 to S650 are repeated. This loop iterates until the matching result indicates that the difference between the predicted dose distribution and the measured dose distribution is small enough. In this case, a beam distribution obtained finally is the target beam distribution. The following description of FIG. 10 provides an example in detail about how to determine a target beam distribution of a beam to be modeled based on a matching result between a predicted dose distribution and a measured dose distribution, which is not described again in this embodiment of the present disclosure.

In the embodiments of the present disclosure, a complex physical modeling issue is converted into a structured and automatically executed mathematical optimization issue by using the foregoing parameterization and iterative optimization procedure. By adjusting parameters of the one-dimensional radial function on discrete sampling points instead of directly operating an entire two-dimensional distribution greatly reduces dimensionality of an optimization space and difficulty of manual intervention. Based on this, in this embodiment of the present disclosure, a deviation between an initial model and a real beam is corrected, finally ensuring that the target beam distribution determined based on the above0mentioned methods has both mathematical simplicity and physical authenticity.

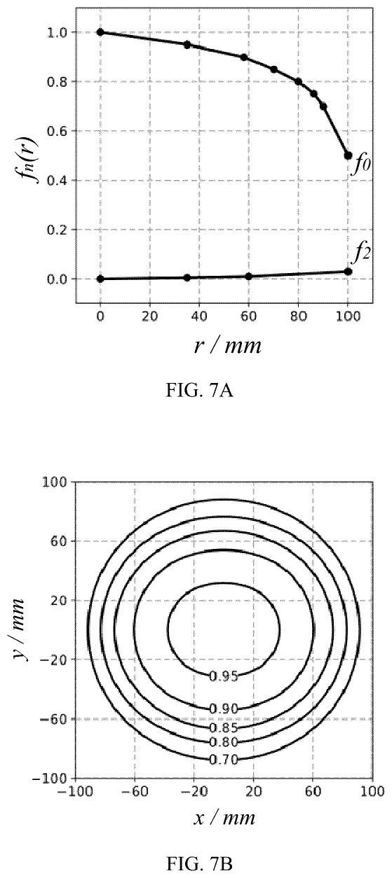

The following describes, with reference to FIG. 7A to FIG. 9B, beam modeling results obtained based on the beam distribution determination method mentioned in the foregoing embodiments.

FIG. 7A and FIG. 7B are schematic diagrams of beam modeling results according to an embodiment of the present disclosure. As shown in FIG. 7A and FIG. 7B, FIG. 7A and FIG. 7B show how to model an ellipse-shaped beam distribution using the beam distribution determination method mentioned in the embodiments of the present disclosure.

FIG. 7A shows two adjusted one-dimensional radial functions ƒ₀(r) and ƒ₂(r). Therein, ƒ₀(r) represents an axisymmetric fundamental component of a beam distribution, and ƒ₂(r) represents a second-order angular frequency component into which elliptic distortion is introduced. By adjusting a shape of ƒ₂(r), a degree of elliptical eccentricity may be controlled.

FIG. 7B shows a two-dimensional beam distribution obtained after two one-dimensional radial functions in FIG. 7A are substituted into a formula of ƒ(r, θ)=ƒ₀(r)+ƒ₂(r)cos(2θ). As shown in the result, the distribution is clearly ellipse-shaped, and a long axis of the ellipse is parallel to an x-axis (because a phase angle 42 in the formula is set to 0°). This figure shows that only a combination of two one-dimensional radial functions can effectively simulate an elliptical beam caused by a factor such as asymmetry of a collimator or an accelerator tube.

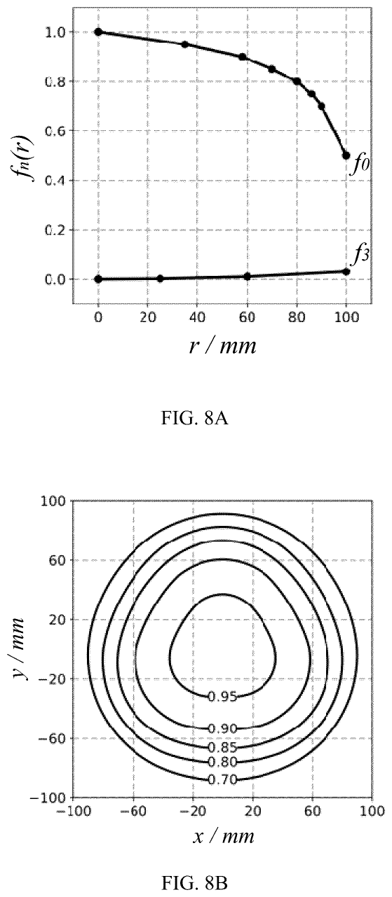

FIG. 8A and FIG. 8B are schematic diagrams of beam modeling results according to another embodiment of the present disclosure. As shown in FIG. 8A and FIG. 8B, FIG. 8A and FIG. 8B show how to model a rounded triangular beam distribution using the beam distribution determination method mentioned in the embodiments of the present disclosure.

FIG. 8A also shows two one-dimensional radial functions ƒ₀(r) and ƒ₃(r). Therein, ƒ₀(r) represents an axisymmetric fundamental component of a beam distribution, and ƒ₃(r) represents a one-dimensional radial function of a third-order angular frequency component. Morphology of ƒ₃(r) determines distinctness and positions of three angles in the beam distribution.

FIG. 8B shows a two-dimensional distribution generated according to a formula of ƒ(r,θ)=ƒ₀(r)+ƒ₃(r) cos(3(θ−90°)). Because a third-order term is selected and a phase angle ψ₃=90° is set, the generated beam distribution exhibits three distinct and smooth angles, which are located in directions of 90°, 210°, and 330°, respectively. This result indicates successful simulation of a rounded triangular beam shape that may arise from factors such as energy-dispersed target irradiation of an electron beam after magnetic field deflection.

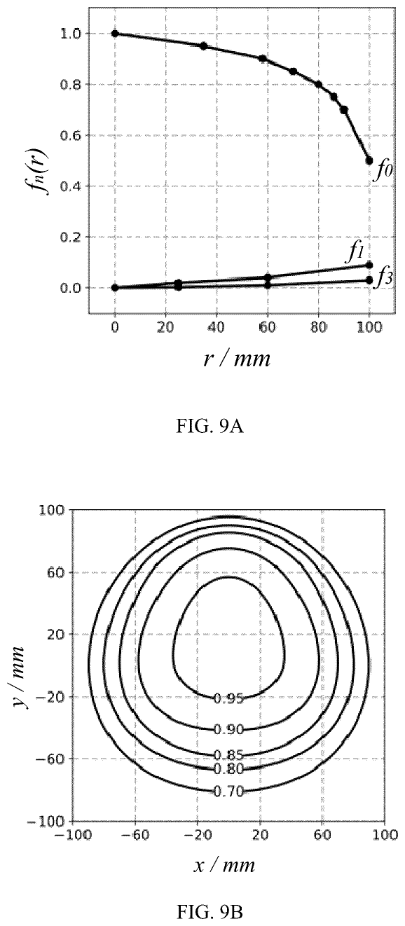

FIG. 9A and FIG. 9B are schematic diagrams of beam modeling results according to still another embodiment of the present disclosure. As shown in FIG. 9A and FIG. 9B, FIG. 9A and FIG. 9B show how to model a pebble-shaped beam distribution using the beam distribution determination method mentioned in the embodiments of the present disclosure.

FIG. 9A shows three one-dimensional radial functions ƒ₀(r), ƒ₁(r), and ƒ₃(r) that are combined into a pebble-shaped distribution. Therein, ƒ₀(r) represents a fundamental axisymmetric component of a beam distribution, ƒ₁(r) is a first-order component, and ƒ₃(r) is a third-order component. ƒ₁(r) is key to formation of an asymmetric egg tip.

FIG. 9B shows a two-dimensional distribution generated according to a formula of ƒ(r,θ)=ƒ₀(r)+ƒ₁(r) cos(θ−90°)+ƒ₃(r) cos[3 (θ−90°)]. By adjusting both the first-order component and the third-order component and setting their phase angles to 90°, a generated beam distribution forms an obvious tip in a positive direction of a y-axis, and overall appears a typical pebble shape. This indicates that the beam distribution determination method mentioned in the embodiments of the present disclosure also has a good description capability for complex beam shapes superimposed with a plurality of distortions.

With reference to FIG. 10, the following further describes how to determine a target beam distribution of a beam to be modeled based on a matching result.

FIG. 10 is a schematic flowchart of determining a target beam distribution according to an embodiment of the present disclosure. The embodiment shown in FIG. 10 is extended based on the embodiment shown in FIG. 5. The following focuses on a difference between the embodiment shown in FIG. 10 and the embodiment shown in FIG. 5, and similarities are not described again.

As shown in FIG. 10, in some embodiments, the determining the target beam distribution of the beam to be modeled based on a matching result between the predicted dose distribution and the measured dose distribution includes the following steps.

Step S1010: determining, based on the matching result, whether the predicted dose distribution and the measured dose distribution meet a consistency condition. If the predicted dose distribution and the measured dose distribution meet the consistency condition, step S1020 is performed. If the predicted dose distribution and the measured dose distribution do not meet the consistency condition, step S1030 is performed.

For example, the consistency condition is a quantization criterion used to determine a degree of matching between the predicted dose distribution and the measured dose distribution. For example, the consistency condition includes that a root mean square error between the predicted dose distribution and the measured dose distribution is less than a first threshold (for example, ≤2%) and/or when a gamma analysis algorithm is used, a gamma pass rate is higher than a second threshold (for example, ≥95%). It may be understood that the foregoing consistency condition is merely an example, and may be specifically set according to an actual requirement.

Step S1020: determining an initial beam distribution as a target beam distribution.

Step S1030: performing iterative adjustment to a part or all of the at least one basis function iteratively.

That is, if the predicted dose distribution and the measured dose distribution are determined to meet the consistency condition based on the matching result, the initial beam distribution is determined as the target beam distribution. If the predicted dose distribution and the measured dose distribution do not meet the consistency condition, iterative adjustment is performed to a part or all of the at least one basis function. For example, a value of a one-dimensional radial function in the at least one basis function at its discrete sampling point may be adjusted.

Then, a new beam distribution is regenerated based on an updated basis function, and a subsequent dose distribution prediction and matching comparison procedure is repeated, until it is determined, based on the matching result, that the predicted dose distribution and the measured dose distribution meet the consistency condition. Then, the initial beam distribution when the consistency condition is met is determined as the target beam distribution.

In this embodiment of the present disclosure, by introducing the consistency condition as an iteration termination condition, not only it is ensured that a final model has verified high accuracy that can match measured data, but also subjective human interference and uncertainty in a modeling process is significantly reduced, thereby providing an efficient, reliable, and repeatable beam modeling solution for clinical application.

Specific implementations of determining at least one basis function based on the measured dose distribution and determining the target beam distribution based on the at least one basis function are described above. In the foregoing embodiments, only dependence of the beam distribution on coordinates is described. However, for some dose calculation algorithms, the beam distribution may be a joint distribution, such as ƒ(E, r, θ) or ƒ(E, vₓ, v_{y}, r, θ), of particle energy E, velocities vₓ and v_{y}, and coordinates r and θ. In this case, division of intervals may be first performed based on energy and/or velocities of particles, and then modeling is performed within each interval by using the beam distribution determination method mentioned in the embodiments of the present disclosure.

Specifically, FIG. 11 is a schematic flowchart of a beam distribution determination method according to another embodiment of the present disclosure. The embodiment shown in FIG. 11 is extended based on the embodiment shown in FIG. 2. The following focuses on a difference between the embodiment shown in FIG. 11 and the embodiment shown in FIG. 2, and similarities are not described again.

As shown in FIG. 11, before the determining the at least one beam distribution component based on the measured dose distribution of the beam to be modeled (step S201), the beam distribution determination method may further include the following steps.

Step S1110: dividing the beam distribution of the beam to be modeled into a plurality of intervals based on energy and/or velocities of particles. In this way, a target beam distribution of the beam to be modeled in each interval can be generated for the beam to be modeled in the interval.

The energy of the particles is energy of the particles forming the beam to be modeled. The energy directly affects a penetration capability, dose deposition efficiency, and a spatial distribution law of a beam. The velocities of the particles refer to speeds at which the particles move for forming the beam, and are a core parameter representing a state of particle motion.

In this step, the particles in the beam to be modeled are first classified according to the energy and/or the velocities of the particles. Specifically, particle energy spectrum and/or velocity distribution information of the particles are first obtained. Subsequently, a distribution of the beam to be modeled is decomposed into a plurality of sub-beam distributions according to a preset division rule, where each sub-beam distribution corresponds to one energy and/or speed interval, that is, each interval corresponds to one independent beam to be modeled.

In the case of division by energy, the beam may be divided into a plurality of equally spaced energy intervals according to an energy magnitude. In the case of division by velocity, the beam may be divided into a plurality of equally spaced velocity intervals according to a velocity magnitude. In the case of division by both energy and velocity, particles whose energy and velocity meet a specific correspondence may be divided into a same interval, so as to ensure that energy and velocity characteristics of particles in each interval are consistent.

Then, a subsequent modeling operation is separately performed on the beam to be modeled in each interval, to obtain a target beam distribution of the beam to be modeled in the interval.

In this embodiment of the present disclosure, a complex beam distribution including a plurality of types of particle energy and/or velocities into a plurality of intervals. A modeling procedure is performed for each interval, to generate the target beam distribution corresponding to each interval. The interval-based modeling approach is adaptable to a joint distribution scenario of particle energy, velocities, and coordinates, to extend an application scope of the beam distribution determination method mentioned in the embodiments of the present disclosure.

In addition, in some cases, a center of a beam still exhibits circular symmetry, while only a region near a beam edge presents a non-circular shape. For example, a photon beam produced by target irradiation is circular, but when it passes through a rectangular collimator, an edge of the beam becomes ellipse-shaped. To adapt the beam distribution determination method mentioned in this embodiment of the present disclosure to this case, a basis function needs to be adjusted accordingly.

Specifically, in some embodiments, a part or all of basis functions include a one-dimensional radial function that is zero at the center of the beam to be modeled and is non-zero at the edge of the beam to be modeled. In the basis function, for any one-dimensional radial function with a radial order greater than 0, (that is, n>0), it is set to zero at the center of the beam and non-zero at the edge of the beam. Because the one-dimensional radial function with n>0 is zero at the center, circular symmetry of a central region is not damaged, and only a shape of a non-central region is modified accordingly, so that a non-edge region exhibits a non-circular characteristic without damaging the circular symmetry of the central region.

For a beam whose center is circularly symmetrical and whose edge is non-circularly distorted, in this embodiment of the present disclosure, the basis function is correspondingly adjusted, so that a target beam distribution obtained finally better aligns with an actual beam distribution, thereby improving accuracy of the target beam distribution in a central region.

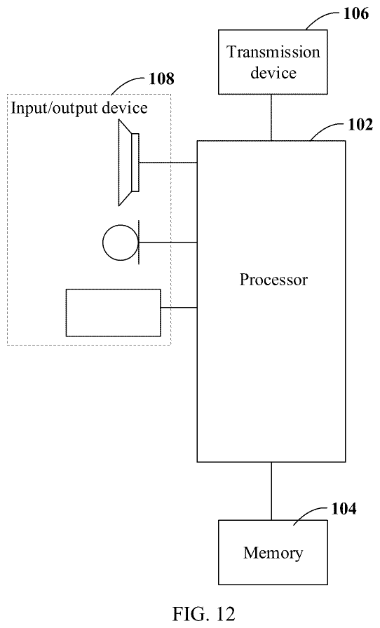

It should be noted that, the method embodiments provided in this embodiment may be executed on terminals, computers, or similar computing devices. For example, when running on a terminal. FIG. 12 is a hardware structure block diagram of a terminal of a beam distribution determination method in this embodiment. As shown in FIG. 12, the terminal may include one or more (only one is shown in FIG. 12) processors 102 and a memory 104 for storing data, where the processor 102 may include, but is not limited to, processing devices such as a Micro Controller Unit (MCU) or a Field-Programmable Gate Array (FPGA). The above terminal may further include a transmission device 106 for communication functions and an input/output device 108. Those skilled in the art may understand that the structure shown in FIG. 12 is only illustrative and does not limit the structure of the above terminal. For example, the terminal may further include more or fewer components than those shown in FIG. 12 or have a different configuration from that shown in FIG. 12.

The memory 104 may be used to store computer programs, such as software programs and modules of application software, such as the computer program corresponding to the beam distribution determination method in this embodiment. The processor 102 executes various functional applications and data processing by running the computer programs stored in the memory 104, thereby implementing the above-mentioned method. The memory 104 may include high-speed random access memory and may further include non-volatile memory, such as one or more magnetic storage devices, flash memories, or other non-volatile solid-state memories. In some instances, the memory 104 may further include memory remotely located relative to the processor 102. These remote memories may be connected to the terminal through a network. Examples of the above network include, but are not limited to, Internet, intranets, local area networks, mobile communication networks, and combinations thereof.

The transmission device 106 is configured to receive or transmit data through the network. The above-mentioned network includes the wireless network provided by a communication carrier of the terminal. In one instance, the transmission device 106 includes a Network Interface Controller (NIC), and may be connected to other network devices through a base station to communicate with the Internet. In one instance, the transmission device 106 may be a Radio Frequency (RF) module, and is used to communicate with the internet wirelessly.

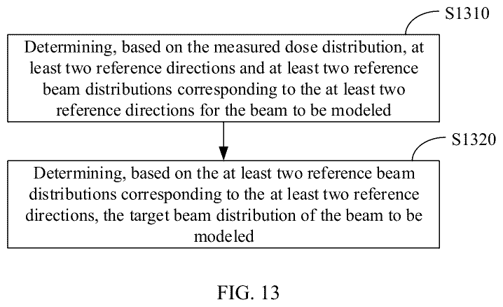

FIG. 13 is a flowchart of a beam distribution determination method in this embodiment. Referring to FIG. 13, the following example illustrates a case where the beam distribution component is a reference beam distribution corresponding to each reference direction.

As shown in FIG. 13, in the embodiment of the present disclosure, the determining the at least one beam distribution component based on the measured dose distribution of a beam to be modeled (step S201), includes step S1310.

Step S1310: determining, based on the measured dose distribution, at least two reference directions and at least two reference beam distributions corresponding to the at least two reference directions for the beam to be modeled.

Specifically, the measured dose distribution may be a two-dimensional measured distribution on a two-dimensional plane, and the two-dimensional plane is a preset plane, i.e., a positional relationship, such as the distance and direction between the plane and a beam source, is preset. In one implementation, the two-dimensional plane is a plane perpendicular to a beam direction and at a preset distance from a beam source. In some other implementation, the two-dimensional plane has an angle with respect to a plane perpendicular to the beam direction.

However, the measured dose distribution is not limited to this. For example, the measured dose distribution may further be a three-dimensional measured distribution in a three-dimensional space, and the three-dimensional space may be a preset spatial volume. In one implementation, the three-dimensional space is a three-dimensional volume at a preset distance from the beam source and surrounding a beam axis direction, or the three-dimensional volume may also be set to other positions.

Before beam modeling, the beam to be modeled is measured, i.e., the two-dimensional measured dose distribution of the beam to be modeled on the one or more two-dimensional planes, or the three-dimensional measured dose distribution of the beam to be modeled in the three-dimensional space is measured. In one implementation, the two-dimensional measured dose distribution or the three-dimensional dose distribution is obtained, a shape of the two-dimensional measured dose distribution or the three-dimensional dose distribution is analyzed, and a reference direction is determined based on the shape. The shape of the two-dimensional measured dose distribution includes, but is not limited to, a shape of isodose lines, a distribution shape of dose points, etc. The shape of the three-dimensional measured dose distribution includes, but is not limited to a shape of isodose surfaces, a distribution shape of dose points, etc. The reference direction may be determined by mathematical operations such as calculating a symmetry axis, a rate of change of the two-dimensional measured dose distribution or the three-dimensional measured dose distribution.

In this embodiment, the determining the target beam distribution of the beam to be modeled based on the at least one beam distribution component (step S202), includes step S1320.

Step S1320: determining, based on the at least two reference beam distributions corresponding to the at least two reference directions, the target beam distribution of the beam to be modeled.

Specifically, for each reference direction, the corresponding reference beam distribution is acquired. The reference beam distribution is an analytical function defined with prior knowledge or a discrete numerical table to express an energy distribution, a particle number distribution, etc., of the beam to be modeled. The reference beam distributions in the reference directions are integrated, values of transition regions between reference directions are calculated, thereby obtaining a complete distribution of the beam, i.e., the target beam distribution. In one implementation, the analytical function includes but is not limited to a one-dimensional function ƒ(r) related to position r or multi-dimensional joint functions ƒ(E,r), ƒ(p,r), ƒ(E, v, r) of energy E, momentum p, velocity v, and position r to express particle flux, energy flux, kerma (kinetic energy released per unit mass) of the beam to be modeled, and other physical quantity distributions. Therefore, a definition of the reference beam distribution varies in different algorithm models. Based on the selected reference beam distribution, corresponding type of the target beam distribution may be synthesized to represent a particle flux distribution or an energy flux distribution of the beam to be modeled, and the like. After the target beam distribution is determined, dose calculation or information analysis may be further conducted based on the target beam distribution, and calculation or analysis results may be applied in various scenarios, such as the debugging of radiotherapy equipment or the design of radiotherapy plans.

In this embodiment, the at least two reference directions are determined for the beam to be modeled, based on the measured dose distribution. A target beam distribution of the beam is determined based on the at least two reference beam distributions corresponding to the at least two reference directions. Therefore, the problem of low beam modeling efficiency is solved. By setting simple beam distributions in several directions and then integrating the beam distributions into the complete target beam distribution, a decomposition of the beam distribution is achieved, thereby reducing workload of the beam modeling and improving calculation speed, and being applicable to various application scenarios involving beams that are circular or deviate from a regular circle.

Here, non-limiting examples of steps S1310 and S1320 are mainly described, with the measured dose distribution being or including the two-dimensional measured dose distribution on the two-dimensional plane as an example. However, it is not limited to this, and the methods described below may also be extended to situation with the measured dose distribution being or including the three-dimensional dose distribution in the three-dimensional space, and details thereof are not repeated herein.

In some embodiments, based on the above step S1320, the determining, based on the at least two reference beam distributions corresponding to the at least two reference directions, the target beam distribution of the beam to be modeled, includes the following steps.

Step S221: acquiring a spatial weight distribution corresponding to the at least two reference directions.

Step S222: integrating, based on the spatial weight distribution, the at least two reference beam distributions on the two-dimensional plane to obtain the target beam distribution on the two-dimensional plane.

Specifically, the spatial weight distribution is a correspondence relationship between each position (x, y) and weight on the two-dimensional plane, and may be represented by a weight function or a discrete weight table. The spatial weight distribution is used to adjust a beam distribution situation when transitioning from one of the reference directions to another of the reference directions, to transition and connect the reference beam distributions.

In some embodiments, based on the above step S1320, the integrating, based on the spatial weight distribution, the at least two reference beam distributions on the two-dimensional plane to obtain the target beam distribution on the two-dimensional, includes the following steps.

Step S310: integrating, based on the spatial weight distribution, the at least two reference beam distributions on the two-dimensional plane to obtain an initial beam distribution on the two-dimensional plane.

Step S320: calculating, based on the initial beam distribution, a predicted dose distribution of the beam to be modeled.

Step S330: adjusting, based on a matching result between the predicted dose distribution and the two-dimensional measured dose distribution, the spatial weight distribution, and re-integrating, based on the spatial weight distribution adjusted, the at least two reference beam distributions on the two-dimensional plane to obtain the target beam distribution.

Specifically, when comparing with measurement results, some specific directions may be selected, and only dose distribution in these directions is compared. Common choices are to compare the dose distribution on x-axis, y-axis, and 45-degree and 135-degree diagonals. Optionally, the dose distribution in the reference direction is selected for comparison.

In some situations, even when beam shape is relatively simple, those skilled in the art may still be unable to construct a good weight function after attempts to make a dose transition shape between the different reference directions consistent with the measurement results. And those skilled in the art may increase the number of the reference directions and reduce a span between adjacent reference directions to improve the shape of the dose distribution when transitioning from one of the reference directions to another of the reference directions.

In this embodiment, based on the decomposition of the beam to be modeled, the accuracy of the target beam distribution is improved through continuous comparison and adjustment.

In some embodiments, the adjusting, based on the matching result between the predicted dose distribution and the two-dimensional measured dose distribution, the spatial weight distribution, and re-integrating, based on the spatial weight distribution adjusted, the at least two reference beam distributions on the two-dimensional plane to obtain the target beam distribution, includes the following steps.

Step S1410: adjusting, based on the matching result between the predicted dose distribution and the two-dimensional measured dose distribution, the at least two reference beam distributions and the spatial weight distribution, and recalculating the predicted dose distribution, until the predicted dose distribution matches the two-dimensional measured dose distribution, to obtain the target beam distribution.

Specifically, along each reference direction, the reference beam distribution ƒᵢ(r) is defined respectively, where i=1, 2, . . . to represent each reference direction. In the formula, r=\sqrt{x²+y²}, where (x, y) is any position vector on the two-dimensional plane.

The weight function wᵢ(x, y) between (x, y) and each reference direction is defined. When the algorithm needs to calculate the beam distribution at any position vector (x, y) on the two-dimensional plane, the following formula is used for calculation:

$$f ( x , y ) = \frac{\sum {w}_{i} ( x , y ) {f}_{i} ( r )}{\sum {w}_{i} ( x , y )} \qquad ( 11 )$$

where, ƒ(x, y) is the target beam distribution, wᵢ(x, y) is the weight function of a certain reference direction, and ƒᵢ(r) is the reference beam distribution of a certain reference direction.

ƒ(x, y) defined in the above steps is used as an input for the dose calculation, and the calculation results are compared with the measurement results. ƒᵢ(r) and wᵢ(x, y) are continuously adjusted until the calculation results are consistent with the measurement results.

In practical applications, ƒᵢ(r) and wᵢ(x, y) may be adjusted simultaneously; alternatively, ƒᵢ(r) may be separated from wᵢ(x, y), and only ƒᵢ(r) or wᵢ(x, y) is adjusted for each time the predicted dose distribution is updated.

In this embodiment, by a dual adjustment of ƒᵢ(r) and wᵢ(x, y), the accuracy of the target beam distribution is further improved, and a requirement for selecting the reference beam distribution in an initial stage is reduced. For example, in a common situation of irregular beam distribution, a center of the irregular beam is still circular symmetry, while only near an edge of the beam, the beam to be modeled exhibits a non-circular shape. If the reference beam distribution set in the initial stage cannot accurately construct the target beam distribution, during an adjustment stage, ƒᵢ(r) in each direction is made equal when r is close to a beam center, while ƒᵢ(r) in each direction may exist differences when r is close to a beam edge. Thus, in subsequent adjustments, the reference beam distribution is gradually optimized, thereby making a calculation process more flexible and simpler.

In the above instance, r is defined as a radial distance from any point (x, y) on the plane to the beam center, but in actual use, a coordinate origin used to define r may also be chosen as points other than the beam center.

In some embodiments, the reference direction determined includes at least two directions determined based on the symmetry axis.

Specifically, the common beam distribution includes an ellipse shape, a pebble shape, and the like. The ellipse shape is not necessarily a mathematically strictly defined ellipse, but broadly refers to various shapes where one axis is stretched and at least one orthogonal axis is flattened. The pebble shape may broadly refer to various shapes where a dose distribution increases along one direction while the dose distribution decreases along the opposite direction. The ellipse shape, the pebble shape, and the other beam distribution may be approximated as standard ellipses, with a long axis as one reference direction, a short axis as another reference direction, or based on an existing symmetry axis, a direction pointing forward on symmetry axis as one reference direction and a direction pointing backward as another reference direction.

In some embodiments, the reference direction determined includes at least two directions with different rates of change in the measured dose distribution.

Specifically, for a non-standard ellipse shape, pebble shape, or other irregular beam distributions, the rate of change of beam distribution of the two-dimensional measured dose distribution or the three-dimensional measured dose distribution along various directions may be calculated, and directions with different rates of change are used as the reference direction.

In some embodiments, the reference direction determined may include at least one direction determined based on the symmetry axis and at least one direction determined based on the rate of change. A more suitable reference direction may be determined based on an actual condition of the beam.

In some embodiments, the spatial weight distribution satisfies a preset requirement, and the preset requirement includes: the spatial weight distribution is applied to transition regions between the respective reference directions.

Specifically, since a purpose of setting the spatial weight distribution is to control the shape of the dose distribution when transitioning from one of the reference directions to another of the reference directions, specific values on the reference direction are determined by the reference beam distribution. Therefore, when acquiring or constructing the spatial weight distribution, it is necessary to ensure that the respective spatial weight distributions are not applied to the reference direction but only to the transition regions, even if ƒₓ(r) is taken on the x-axis of ƒ(x, y) and ƒ_{y}(r) is taken on the y-axis of ƒ(x, y). On the other hand, the spatial weight distribution is required to ensure a smooth transition of ƒ(x, y) between the respective reference directions.

The following describes and illustrates this embodiment through optional examples.

To improve a speed of the dose calculation, clinically used dose calculation software abstracts and simplifies a treatment head. In the dose calculation, algorithms typically omit the generation process of the beam in the treatment head and initial few processes the beam undergoes in the treatment head, such as the beam passing through a primary collimator, a flattening filter, an ionization chamber. The algorithm does not simulate these processes. Instead, by modeling, the algorithm uses empirical analytical functions or numerical tables to directly describe the distribution of the beam after undergoing the above-mentioned processes. After the above-mentioned beam distribution is obtained by modeling, the algorithm only needs to simulate subsequent processes the beam undergoes, typically including shaping and hardening of the beam as it passes through a tungsten collimator and a multi-leaf collimator, as well as an energy deposition process after entering the human body.

Modeling the beam distribution, i.e., calculating the beam distribution, is a key step in the above-mentioned process.

Since the beams generated by the treatment head are usually circular, when modeling the beam, the beam is often assumed to have circular symmetry, and a one-dimensional function ƒ(r) is correspondingly set to describe the beam distribution with circular symmetry. Depending on the different algorithms, ƒ(r) here may refer to the particle flux, the energy flux, the kerma of the beam, etc., or other physical quantities of the particles, such as the energy E, the momentum p, or the velocity v, in a joint distribution with position r, i.e., ƒ(E, r), ƒ(p,r), ƒ(E, v, r). A dependence relationship of ƒ on r may be given by empirical analytical formulas or the discrete numerical tables. Here, ƒ(r) is defined on a specific two-dimensional plane perpendicular to a beam center line. In addition, r is the radial distance from any point (x, y) on this plane to the beam center, i.e., r=\sqrt{x²+y²}.

To model the beam, tools such as water tanks, phantoms, films, and flat panels are typically used to measure the dose distribution of the beam in these materials. These dose distributions are essentially three-dimensional, but usually the dose distribution of only a subset of points, lines, or surfaces is selected to model the beam. For example, a dose distribution curve along a depth on the beam center line may be used to model an energy spectrum of the beam, while the dose distribution on the plane perpendicular to the beam direction may be used to model the beam shape.

When modeling ƒ(r), specific planes at certain depths and perpendicular to the beam direction are usually selected. Then, ƒ(r) is used as the input for the algorithm to calculate the dose distribution on these planes, and the dose distribution is then compared with the measurement results. ƒ(r) is continuously adjusted until the calculation results are consistent with the measurement results.

Here, when ƒ(r) is given in the form of an analytical formula, the modeling process involves adjusting parameters in the analytical formula; and if ƒ(r) is given in the form of a numerical table, the modeling process involves adjusting each value in the table.

Undoubtedly, since the beam described by ƒ(r) has circular symmetry, the dose distribution calculated in a uniform material may also has circular symmetry. Specifically, when comparing with the measurement results, people usually do not compare the dose distribution over the entire two-dimensional plane, but select some specific directions and only compare the dose distribution in these directions. Common choices are to compare the dose distribution on the x-axis, the y-axis, and 45-degree and 135-degree diagonals.

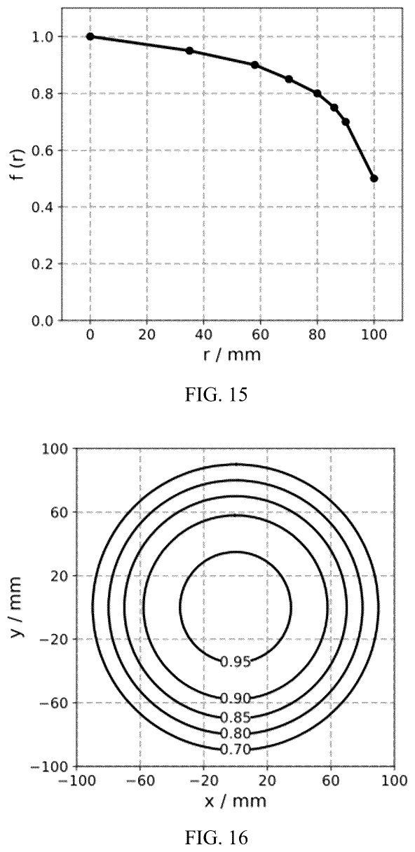

Once ƒ(r) is given, ƒ(r) may be used as the input for the algorithm to perform the dose calculation. When the algorithm needs to calculate a value of ƒ at any position (x, y) on the two-dimensional plane, it only needs to calculate r=\sqrt{x²+y²} and then substitute it into ƒ(r) for calculation. As an example, FIG. 15 shows a one-dimensional function ƒ(r), while FIG. 16 shows a restored two-dimensional distribution from this function, i.e., ƒ(x, y)=ƒ(\sqrt{x²+y²}).

The above is a description of a modeling process for a circular beam. For the non-circular beam, the following method is further proposed in the optional embodiment.

1. Two or a few specific directions, referred to as reference directions, are selected on a two-dimensional plane. Then, a beam distribution is modeled along these reference directions.

2. For any vector on the two-dimensional plane, a weight function between this vector and each of the above-mentioned reference directions is defined. Then, these weight functions are used to synthesize the beam distribution on each reference direction into the beam distribution on the entire two-dimensional plane.

The non-circular beam modeling method is further proposed in this optional embodiment. The description of the beam distribution of non-circular is achieved by extending one-dimensional function ƒ(r) to the beam distribution in two or a few specific directions, and defining the weight function between any position vector and these directions. In this optional embodiment, when adjusting the beam distribution on each reference direction, it is only required to compare the dose distribution on that reference direction.

Next, as two optional examples, a description of the beam distribution of ellipse-shaped and pebble-shaped is demonstrated in this optional embodiment.

For the beam distribution of the ellipse-shaped, we assume that a long axis of the ellipse-shaped is an x-axis on the two-dimensional plane, while a short axis is a y-axis on the two-dimensional plane. Based on a symmetry of the ellipse-shaped, we may easily select the long axis and the short axis of the ellipse-shaped as the reference directions. Since the ellipse-shaped is symmetric about the long axis and the short axis in horizontal and vertical directions, respectively, we only need to model the beam distribution in a positive direction of the x-axis and a positive direction of the y-axis, denoted as ƒₓ(r) and ƒ_{y}(r) respectively. To synthesize ƒₓ(r) and ƒ_{y}(r) into a two-dimensional beam distribution ƒ(x, y), we define the weight functions for any position vector (x, y) on the two-dimensional plane with respect to the two reference directions, denoted as wₓ(x, y) and w_{y}(x, y) respectively. We set functional forms of the two functions as wₓ(x, y)=x² and w_{y}(x, y)=y², respectively, and then we may obtain the beam distribution on the two-dimensional plane according to the above formula (11).

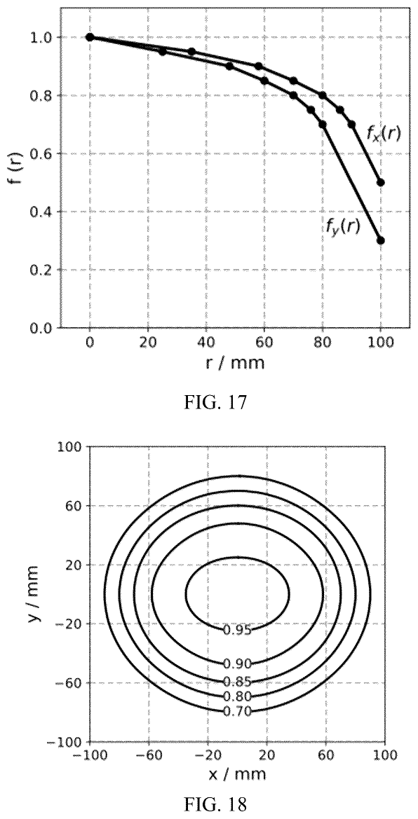

Here, when constructing the two weight functions, those skilled in the art need to rely on experience to select the functional forms to meet certain specific requirements. For example, the two weight functions may make ƒ(x, y) take ƒₓ(r) on the x-axis and ƒ_{y}(r) on the y-axis. Additionally, the two weight functions may make ƒ(x, y) symmetric about the x-axis and the y-axis. Furthermore, when ƒ(x, y) transitions between the x-axis and the y-axis, a shape of ƒ(x, y) may be naturally smooth to conform to perception of the ellipse-shaped, and make dose calculation results consistent with measurement results. FIG. 17 shows a specific adjustment of ƒₓ(r) and ƒ_{y}(r), while FIG. 18 shows a two-dimensional distribution synthesized using the above method.

For a beam distribution of pebble-shaped, it is assumed that a central axis of the pebble-shaped is a y-axis on a two-dimensional plane, that is, the pebble-shaped is asymmetric in the positive and negative directions of the y-axis but symmetric about the y-axis. Based on this symmetry, the positive and negative directions of the y-axis are selected as reference directions, and the beam distribution on the two reference directions is denoted as ƒ₊(r) and ƒ₋(r).

To synthesize ƒ₊(r) and ƒ₋(r) into the two-dimensional beam distribution ƒ(x, y), the present disclosure defines the weight functions for any position vector (x, y) on the two-dimensional plane with respect to the two reference directions, denoted as w₊(x, y) and w₋(x, y) respectively. The functional forms of the two functions are set as w₊(x, y)=(r+y)² and w₋(x,y)=(r−y)², respectively, and then the beam distribution on the two-dimensional plane according to the formula (11) may be obtained.

Here, the functional forms of the two weight functions meet certain specific requirements. For example, the two weight functions may make ƒ(x, y) take ƒ₊(r) on a positive half-axis of the y-axis and ƒ₋(r) on a negative half-axis of the y-axis. Additionally, the two weight functions may make ƒ(x, y) symmetric about the y-axis. Furthermore, when transitioning between the positive and negative half-axes of the y-axis, a shape of ƒ(x, y) may be naturally smooth to conform to perception of the pebble-shaped, and make the dose calculation results consistent with measurement results.

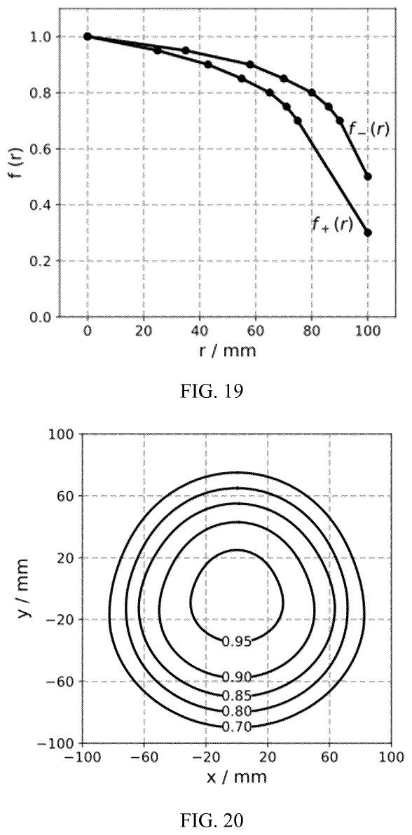

FIG. 19 shows a specific adjustment of ƒ₊(r) and ƒ₋(r), while FIG. 20 shows a two-dimensional distribution synthesized using the above method.

The above examples only demonstrate a method of performing modeling on a beam distribution of ellipse-shaped and pebble-shaped. However, those skilled in the art with a certain level of experience may apply the method proposed in the present disclosure to perform modeling on the beam distribution of other shapes with a relatively simple process of work.

Those skilled in the art may understand that an algorithm model requires a trade-off between the complexity of the model and the accuracy of the algorithm. Therefore, when the method of the present disclosure is applied to a beam modeling, even when a calculated dose distribution does not completely match measurement results, as long as a difference is within an acceptable range, a validity of this method may be accepted.

A beam modeling method proposed in this optional embodiment is simple, requires minimal modeling workload for a non-circular beam, and with a higher operability.

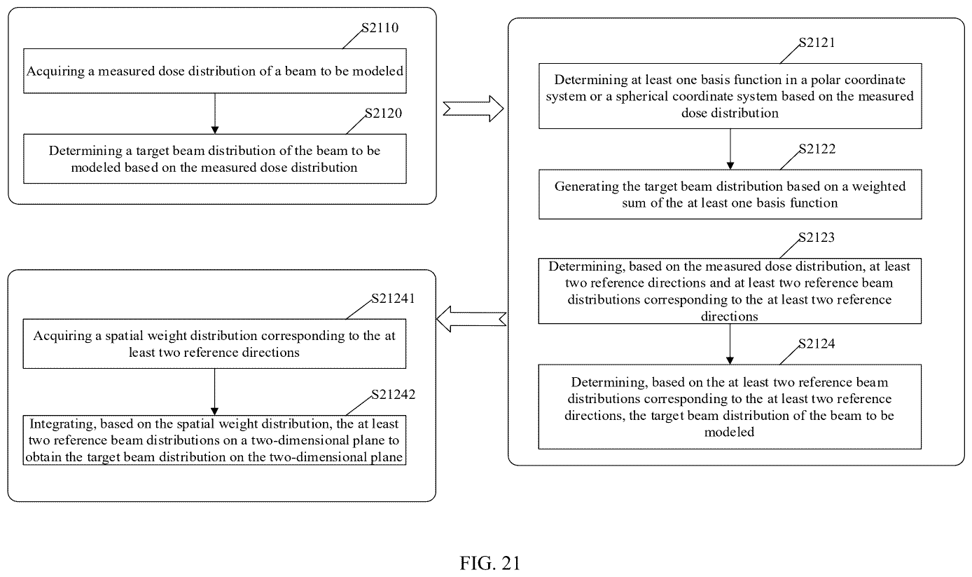

Another beam distribution determination method mentioned in the embodiments of the present disclosure will be described with reference to FIG. 21 in the following.

FIG. 21 is a schematic flowchart of a beam distribution determination method according to still another embodiment of the present disclosure. As shown in FIG. 21, the beam distribution determination method provided by this embodiment of the present disclosure include the following steps.

Step S2110: acquiring a measured dose distribution of a beam to be modeled.

Step S2120: determining a target beam distribution of the beam to be modeled based on the measured dose distribution. Therein, the target beam distribution includes at least one one-dimensional beam distribution component.

This embodiment of the present disclosure achieves the modeling of high-dimensional (e.g., 2D or 3D) functional representations of the measured dose distribution by modeling and combining a finite number of one-dimensional beam components. Consequently, the computational load for modeling is significantly reduced, leading to a substantial improvement in modeling efficiency.

Still referring to FIG. 21, in some embodiments, the determining the target beam distribution of the beam to be modeled based on the measured dose distribution (step S2120) includes the following steps.

Step S2121: determining at least one basis function in a polar coordinate system or a spherical coordinate system based on the measured dose distribution. Each of a part or all of the at least one basis function includes a one-dimensional radial function and an angle function.

Step S2122: generating the target beam distribution based on a weighted sum of the at least one basis function.

In this embodiment of the present disclosure, by transforming the measured dose distribution into a coordinate system (the polar coordinate system or the spherical coordinate system) that aligns with its geometric features and decomposing it into a finite number of basis functions composed of one-dimensional radial functions and angle functions, a problem of modeling a two-dimensional or three-dimensional distribution is simplified to modeling a set of one-dimensional radial functions by by reducing dimensions. This fundamentally reduces dimensionality of parameters to be optimized. Furthermore, this embodiment reconstructs the target beam distribution through a weighted sum of the basis functions, which mathematically provides a prerequisite for accurate fitting of a complex asymmetric dose distribution.

Still referring to FIG. 21, in some embodiments, the determining the target beam distribution of the beam to be modeled based on the measured dose distribution (step S2120) includes the following steps.

Step S2123: determining, based on the measured dose distribution, at least two reference directions and at least two reference beam distributions corresponding to the at least two reference directions.

Step S2124: determining, based on the at least two reference beam distributions corresponding to the at least two reference directions, the target beam distribution of the beam to be modeled.

In this embodiment of the present disclosure, a plurality of reference directions and reference beam distributions corresponding to the plurality of reference directions are utilized to extract characteristic information along different reference directions from the measured dose distribution. The target beam distribution is then synthesized by fusing the characteristic information from the different reference directions. This approach not only enhances modeling efficiency but also ensures modeling accuracy.

Still referring to FIG. 21, in some embodiments, the determining, based on the at least two reference beam distributions corresponding to the at least two reference directions, the target beam distribution (step S2124) includes the following steps.

Step S21241: acquiring a spatial weight distribution corresponding to the at least two reference directions.

Step S21242: integrating, based on the spatial weight distribution, the at least two reference beam distributions on a two-dimensional plane to obtain the target beam distribution on the two-dimensional plane.

In this embodiment of the present disclosure, the modeling of a continuous distribution on a two-dimensional plane is converted into adjustment and weighted fusion of a finite number of one-dimensional beam components by utilizing spatial weight distribution. This avoids the need for dense sampling and cumbersome global parameter adjustments on the two-dimensional plane, fundamentally reducing complexity of modeling and the number of parameters required.

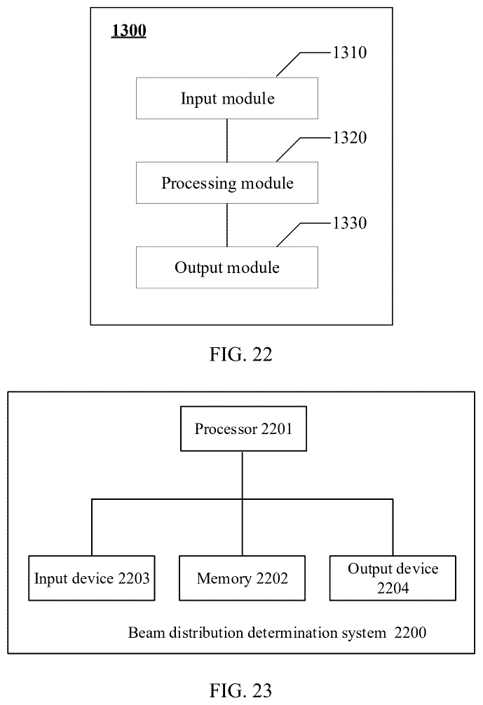

The method embodiments of the present disclosure are described in detail with reference to FIG. 2 to FIG. 21. An apparatus embodiment of the present disclosure is described in detail with reference to FIGS. 22 to 24. In addition, it should be understood that the descriptions of the method embodiments correspond to descriptions of the apparatus embodiments, and therefore, for parts that are not described in detail, reference may be made to the foregoing method embodiments.

FIG. 22 is a schematic structural diagram of an intelligent agent according to an embodiment of the present disclosure. As shown in FIG. 22, an intelligent agent 1300 includes an input module 1310, a processing module 1320, and an output module 1330.

The input module 1310 is configured to obtain a measured dose distribution of a beam to be modeled. Optionally, the input module 1310 is a primary link for the intelligent agent 1300 to interact with the outside, and the input module 1310 can accurately receive input data of a user.

The processing module 1320 is configured to: determine at least one beam distribution component based on a measured dose distribution of a beam to be modeled, where dimensionality of parameter space required to parametrically model the at least one beam distribution component is lower than dimensionality of parameter space required to parametrically model a beam distribution of the beam to be modeled; and determine a target beam distribution of the beam to be modeled based on the at least one beam distribution component. Alternatively, the processing module 1320 is configured to: acquire a measured dose distribution of a beam to be modeled; and determine a target beam distribution of the beam to be modeled based on the measured dose distribution, where the target beam distribution includes at least one one-dimensional beam distribution component.

The output module 1330 is configured to output the target beam distribution of the beam to be modeled.

The intelligent agent in this embodiment of the present disclosure can simply and efficiently improve a degree of intelligence, and improve flexibility and versatility.

An embodiment of a beam distribution determination system of the present disclosure is described in detail below.

FIG. 23 is a schematic structural diagram of a beam distribution determination system according to an embodiment of the present disclosure. As shown in FIG. 23, the beam distribution determination system 2200 includes one or more processors 2201 and a memory 2202.

The processor 2201 may be a Central Processing Unit (CPU) or other form of processing unit with data processing and/or instruction execution capabilities, and can control other components in the beam distribution determination system 2200 to perform desired functions.

The memory 2202 may include one or more computer program products, which may include various forms of computer-readable storage medium, such as volatile memory and/or non-volatile memory. The volatile memory may include, for example, a Random Access Memory (RAM) and/or cache. The non volatile memory may include a Read-Only Memory (ROM), hard disk, flash memory, and the like. One or more computer program instructions can be stored on a computer-readable storage medium, and the processor 2201 is configured to execute the program instructions to implement the methods and/or other desired functions of the embodiments disclosed above. Various contents such as measured dose distribution, target beam distribution, and the like can also be stored in computer-readable storage medium.

In one embodiment, the beam distribution determination system 2200 may further include an input device 2203 and an output device 2204, which are interconnected through a bus system and/or other forms of connection mechanisms (not shown in the figure).

The input device 2203 may include, for example, a keyboard, a mouse, and so on.

The output device 2204 is configured to output various information to external devices, including the target beam distribution, etc. The output device 2204 may include, for example, a display, a speaker, a printer, as well as a communication network and a remote output device connected thereto, and so on.

Of course, for simplicity, only some components related to the present disclosure in the beam distribution determination system 2200 are shown in FIG. 23, and components such as buses, input/output interfaces, and the like are omitted. In addition, depending on the specific application situations, the beam distribution determination system 2200 may further include any other suitable components.

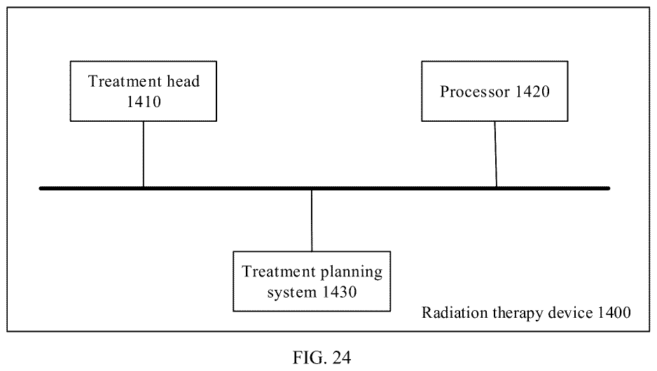

In some embodiments, the beam distribution determination system may be a radiation therapy device, which will be described in the following with reference to FIG. 24. FIG. 24 is a schematic structural diagram of a radiation therapy device according to an embodiment of the present disclosure. As shown in FIG. 24, the radiation therapy device 1400 includes a treatment head 1410, a processor 1420, and a treatment planning system 1430. Components of the radiation therapy device 1400 can communicate with each other via a bus.

The treatment head 1410 is beam generation apparatus of the radiation therapy device 1400, and has a core function of generating a radiation therapy beam that meets a treatment requirement. An accelerator tube, a target material, a primary collimator, an equalizer, and the like are integrated in the treatment head 1410. By adjusting parameters such as accelerator tube energy and collimator opening/closing degree, beams with different energy and initial shapes can be output. In a beam modeling phase, the treatment head 1410 is configured to output a beam to be modeled based on a control instruction of the processor 1420. In a treatment execution phase, the treatment head 1410 is configured to output a corresponding beam based on a planning parameter of the treatment planning system 1430.

The processor 1420 may perform the beam distribution determination method mentioned in the embodiments of the present disclosure. Specifically, the processor 1420 can implement the following steps: determining at least one beam distribution component based on a measured dose distribution of a beam to be modeled, where dimensionality of parameter space required to parametrically model the at least one beam distribution component is lower than dimensionality of parameter space required to parametrically model a beam distribution of the beam to be modeled; and determining a target beam distribution of the beam to be modeled based on the at least one beam distribution component, so as to provide a core input for dose calculation. Alternatively, the processor 1420 can implement the following steps: acquiring a measured dose distribution of a beam to be modeled; and determining a target beam distribution of the beam to be modeled based on the measured dose distribution, where the target beam distribution includes at least one one-dimensional beam distribution component.

The treatment planning system 1430 is configured to perform patient dose calculation and/or treatment plan design based on the target beam distribution of the radiation therapy beam. Specifically, the treatment planning system 1430 receives the target beam distribution generated by the processor 1420, implements dose calculation and treatment plan optimization based on the target beam distribution with reference to information such as a location, a size, and a shape of a target region of patient radiation therapy, and generates a planning parameter of an executable treatment plan, so that the treatment head 1410 outputs a corresponding beam for treatment based on the planning parameter.

In addition, the radiation therapy device 1400 may further include a memory.

The memory may store data and/or instructions, and the memory may store data and/or instructions that may be executed by the processor 1420 or are used to perform the exemplary method in the present disclosure. Optionally, the memory may further store the target beam distribution, dose calculation, and a treatment plan result generated by the processor 1420 and the treatment planning system 1430. Optionally, the memory may be a Read-Only Memory (ROM), a static memory, a dynamic memory, a Random Access Memory (RAM), or the like. The memory is configured to store a computer program. When the computer program stored in the memory is executed by the processor 1420, the processor 1420 and the treatment planning system 1430 are configured to perform steps of the beam distribution determination method in the embodiments of the present disclosure. In some embodiments, the memory may be integrated into the radiation therapy device 1400.

Optionally, the processor 1420 may use a general-purpose Central Processing Unit (CPU), a micro-processor, an Application-Specific Integrated Circuit (ASIC), a Graphics Processing Unit (GPU), or one or more integrated circuits, to execute a related program to achieve functions required to be performed by units in the beam distribution determination apparatus in the embodiments of the present disclosure.

Alternatively, the processor 1420 may be an integrated circuit chip, with a signal processing capability. In an implementation process, steps of the beam distribution determination method in the present disclosure may be completed by using an integrated logic circuit of hardware in the processor 1420 or instructions in a form of software. The processor 1420 may alternatively be a general-purpose processor, a Digital Signal Processor (DSP), an Application-Specific Integrated Circuit (ASIC), a Field Programmable Gate Array (FPGA) or another programmable logic device, a discrete gate or transistor logic device, a discrete hardware component, or the like. The processor 1420 may implement or execute the methods, steps, and logical block diagrams disclosed in the embodiments of the present disclosure. The general-purpose processor may be a microprocessor, or the processor may be any conventional processor or the like. The steps of the methods disclosed with reference to the embodiments of the present disclosure may be directly implemented by a hardware decoding processor, or may be implemented by a combination of hardware and software modules in a decoding processor. The software module may be located in a mature storage medium in the art, for example, a random access memory, a flash memory, a read-only memory, a programmable read-only memory, an erasable programmable memory, or a register. The storage medium is located in the memory.

The processor 1420 reads information from the memory, and in conjunction with its hardware, performs a function required to be executed by a unit included in the beam distribution determination apparatus in the embodiments of the present disclosure, or executes the beam distribution determination method in method embodiments of the present disclosure. In some embodiments, the processor 1420 or a portion of the processor 1420 may be integrated into the radiation therapy device 1400.

In addition, according to a specific requirement, those skilled in the art should understand that the radiation therapy device 1400 may further include a hardware component that implements another additional function. Besides, those skilled in the art should understand that the radiation therapy device may include only components required for implementing embodiments of the present disclosure, but does not need to include all components shown in FIG. 24.

In addition to the foregoing method, apparatus, and device, an embodiment of the present disclosure may further provide a computer program product, including computer program instructions. When the computer program instructions are run by a processor, steps of the beam distribution determination method provided in the embodiments of the present disclosure are implemented.

The computer program product may be written in any combination of one or more programming languages for executing program code of operations of embodiments of the present disclosure, where the programming languages include object-oriented programming languages such as Java and C++, and further include conventional procedural programming languages such as the “C” language or similar programming languages. The program code may be executed entirely on a user computing device, or partially on a user computing device, or as a separate software package, or partially on the user computing device and partially on a remote computing device, or completely on the remote computing device or a server.

In addition, an embodiment of the present disclosure may also be a computer-readable storage medium, where the computer-readable storage medium stores computer program instructions, and when the computer program instructions are run by a processor, steps of the beam distribution determination method provided in the embodiments of the present disclosure are implemented.

The computer-readable storage medium can be any combination of one or more readable media. The readable medium may be a readable signal medium or a readable storage medium. The readable storage medium may include, for example, but is not limited to, an electrical, magnetic, optical, electromagnetic, infrared, or semiconductor system, apparatus, or component or any combination thereof. A more specific example (non-exhaustive list) of the readable storage medium includes: an electrically connected or portable disk with one or more wires, a hard disk, a Random Access Memory (RAM), a Read-Only Memory (ROM), an Erasable Programmable Read-Only Memory (EPROM or flash memory), an optical fiber, a portable Compact Disc Read-Only Memory (CD-ROM), an optical storage device, a magnetic storage device, or any suitable combination thereof.

A person of ordinary skill in the art may be aware that, units and algorithm steps in examples described in combination with the embodiments disclosed in this specification can be implemented by electronic hardware or a combination of computer software and electronic hardware. Whether the functions are performed by hardware or software depends on particular applications and design constraints of the technical solutions. those skilled in the art may use different methods to implement the described functions for each specific application, but it should not be considered that the implementation goes beyond the scope of the present disclosure.

It may be clearly understood by those skilled in the art that, for the purpose of convenient and brief description, for a detailed working process of the foregoing system, apparatus, and unit, reference may be made to corresponding processes in the foregoing method embodiments, and details are not described herein again.

In several embodiments provided in the present disclosure, it should be understood that the disclosed system, apparatus, and method may be implemented in another manner. For example, the described apparatus embodiments are merely examples. For example, the unit division is merely logical function division and may be other division in actual implementation. For example, a plurality of units or components may be combined or integrated into another system, or some features may be ignored or not performed. In addition, the displayed or discussed mutual couplings or direct couplings or communication connections may be implemented as indirect couplings or communication connections through some interfaces, apparatus or units, and may be implemented in electronic, mechanical, or other forms.

The units described as separate parts may or may not be physically separate, and parts displayed as units may or may not be physical units, and may be located in one location, or may be distributed on a plurality of network units. Some or all of the units may be selected according to actual needs to achieve the objective of the solutions of embodiments.

In addition, functional units in the embodiments of the present disclosure may be integrated into one similar area division unit, or each unit may exist alone physically, or two or more units may be integrated into one unit.

When the functions are implemented in a form of a software functional unit and sold or used as an independent product, the functions may be stored in a computer-readable storage medium. Based on such an understanding, the technical solutions of the present disclosure essentially, or the part contributing to the conventional technology, or some of the technical solutions may be implemented in the form of a software product. The computer software product is stored in a storage medium and includes several instructions for instructing a computer device (which may be a personal computer, a server, a network device, or the like) to execute all or some of the steps of the methods described in the embodiments of the present disclosure. The foregoing storage medium includes: any medium that can store program code, such as a USB flash disk, a removable hard disk, a read-only memory, a random access memory, a magnetic disk, or an optical disc.

The foregoing descriptions are merely specific implementations of the present disclosure, but the scope of protection of the present disclosure is not limited thereto. Any variation or substitution that can be easily conceived by those skilled in the art within the scope of the technology disclosed in the present disclosure should be included within the scope of protection of the present disclosure. Therefore, the protection scope of the present disclosure shall be subject to the protection scope of the claims.

What is claimed is:

1. A beam distribution determination method, comprising:
determining at least one beam distribution component based on a measured dose distribution of a beam to be modeled, wherein dimensionality of parameter space required to parametrically model the at least one beam distribution component is lower than dimensionality of parameter space required to parametrically model a beam distribution of the beam to be modeled; and
determining a target beam distribution of the beam to be modeled based on the at least one beam distribution component.

2. The method according to claim 1, wherein the determining the at least one beam distribution component based on the measured dose distribution of the beam to be modeled comprises:
determining at least one basis function based on the measured dose distribution of the beam to be modeled, the at least one basis function being used to represent a multi-dimensional function corresponding to the beam distribution of the beam to be modeled; and
wherein the determining the target beam distribution of the beam to be modeled based on the at least one beam distribution component comprises:
determining the target beam distribution of the beam to be modeled based on the at least one basis function.

3. The method according to claim 2, wherein the determining the at least one basis function based on the measured dose distribution of the beam to be modeled comprises:
determining at least one basis function in a polar coordinate system based on the measured dose distribution of the beam to be modeled, wherein each of a part or all of the at least one basis function comprises a one-dimensional radial function and an angle function, and the measured dose distribution comprises a two-dimensional measured dose distribution on a two-dimensional plane.

4. The method according to claim 3, wherein the determining the at least one basis function in the polar coordinate system based on the measured dose distribution of the beam to be modeled comprises:
decomposing a multi-dimensional function describing the measured dose distribution into a Fourier series function in the polar coordinate system; and
selecting, based on the measured dose distribution, a function term of a target order in the Fourier series function, to obtain the at least one basis function.

5. The method according to claim 2, wherein the determining the at least one basis function based on the measured dose distribution of the beam to be modeled comprises:
determining at least one basis function in a spherical coordinate system based on the measured dose distribution of the beam to be modeled, wherein each of a part or all of the at least one basis function comprises a one-dimensional radial function and an angle function, and the measured dose distribution comprises a three-dimensional measured dose distribution in a three-dimensional space.

6. The method according to claim 5, wherein the determining the at least one basis function in the spherical coordinate system based on the measured dose distribution of the beam to be modeled comprises:
decomposing a multi-dimensional function describing the measured dose distribution into a spherical harmonic function in the spherical coordinate system; and
selecting, based on the measured dose distribution, a function term of a target order in the spherical harmonic function, to obtain the at least one basis function.

7. The method according to claim 2, wherein the determining the target beam distribution of the beam to be modeled based on the at least one basis function comprises:
parameterizing the at least one basis function; and
determining the target beam distribution of the beam to be modeled based on a sum of the at least one basis function.

8. The method according to claim 1, before the determining the at least one beam distribution component based on the measured dose distribution of the beam to be modeled, further comprising:
dividing the beam distribution of the beam to be modeled into a plurality of intervals based on energy and/or velocity of particles, to generate, for a beam to be modeled in each interval, a target beam distribution of the beam to be modeled in the interval.

9. The method according to claim 1, wherein the determining the at least one beam distribution component based on the measured dose distribution of the beam to be modeled comprises:
determining, based on the measured dose distribution, at least two reference directions and at least two reference beam distributions corresponding to the at least two reference directions for the beam to be modeled; and
wherein the determining the target beam distribution of the beam to be modeled based on the at least one beam distribution component comprises:
determining, based on the at least two reference beam distributions corresponding to the at least two reference directions, the target beam distribution of the beam to be modeled.

10. The method according to claim 9, wherein the measured dose distribution comprises a two-dimensional measured dose distribution on a two-dimensional plane.

11. The method according to claim 10, wherein the determining, based on the at least two reference beam distributions corresponding to the at least two reference directions, the target beam distribution of the beam to be modeled comprises:
acquiring a spatial weight distribution corresponding to the at least two reference directions; and
integrating, based on the spatial weight distribution, the at least two reference beam distributions on the two-dimensional plane to obtain the target beam distribution on the two-dimensional plane.

12. The method according to claim 11, wherein the spatial weight distribution satisfies a preset requirement, and the preset requirement comprises:
the spatial weight distribution is applied to transition regions between the at least two reference directions.

13. The method according to claim 10, wherein the two-dimensional plane comprises a plane perpendicular to a beam direction of the beam to be modeled and at a preset distance from a beam source.

14. The method according to claim 9, wherein the at least two reference directions comprise at least two directions determined based on a symmetry axis, and/or the at least two reference directions comprise at least two directions with different rates of change in the measured dose distribution.

15. A beam distribution determination method, comprising:
acquiring a measured dose distribution of a beam to be modeled; and
determining a target beam distribution of the beam to be modeled based on the measured dose distribution, wherein the target beam distribution comprises at least one one-dimensional beam distribution component.

16. The method according to claim 15, wherein the determining the target beam distribution of the beam to be modeled based on the measured dose distribution comprises:
determining at least one basis function in a polar coordinate system or a spherical coordinate system based on the measured dose distribution, wherein each of a part or all of the at least one basis function comprises a one-dimensional radial function and an angle function; and
generating the target beam distribution based on a weighted sum of the at least one basis function.

17. The method according to claim 15, wherein the determining the target beam distribution of the beam to be modeled based on the measured dose distribution comprises:
determining, based on the measured dose distribution, at least two reference directions and at least two reference beam distributions corresponding to the at least two reference directions; and
determining, based on the at least two reference beam distributions corresponding to the at least two reference directions, the target beam distribution of the beam to be modeled.

18. The method according to claim 17, wherein the determining, based on the at least two reference beam distributions corresponding to the at least two reference directions, the target beam distribution of the beam to be modeled comprises:
acquiring a spatial weight distribution corresponding to the at least two reference directions; and
integrating, based on the spatial weight distribution, the at least two reference beam distributions on a two-dimensional plane to obtain the target beam distribution on the two-dimensional plane.

19. A non-transitory computer-readable storage medium, storing a computer program, wherein when the computer program is executed by a processor, the steps of the beam distribution determination method according to claim 1 are implemented.

20. A beam distribution determination system, comprising:
a processor;
a memory, storing instructions executable by the processor; wherein
the processor is configured to implement the following steps:
determining at least one beam distribution component based on a measured dose distribution of a beam to be modeled, wherein dimensionality of parameter space required to parametrically model the at least one beam distribution component is lower than dimensionality of parameter space required to parametrically model a beam distribution of the beam to be modeled; and
determining a target beam distribution of the beam to be modeled based on the at least one beam distribution component.
